(19) United States
(12) Patent Application Publication
(10) Pub. No.: US 2006/0122402 A1
(43) Pub. Date: Jun. 8, 2006

(54) Manufacturing process for no-donating compounds such as no-donating diclofenac

(76) Inventors: Johan Andersson, Sodertalje (SE); Aldo Belli, Cornate D'Adda (IT); Vincenzo Cannata, Sasso Marconi (IT); Martin Hedberg, Sodertalje (SE); Andreas Palmgren, Sodertalje (SE); Sigrid Schuldei, Sodertalje (SE); Marika Strom, Sodertalje (SE); Marco Villa, Sodertalje (SE)

Correspondence Address: ARENT FOX PLLC, 1050 CONNECTICUT AVENUE, N.W., SUITE 400, WASHINGTON, DC, 20036, US

(21) Appl. No.: 10/527,647

(22) PCT Filed: Sep. 18, 2003
(86) PCT No.: PCT/SE03/01465

§ 371 Date: Aug. 1, 2005

(30) Foreign Application Priority Data
Sep. 20, 2002 (SE) 0202801-7
May 20, 2003 (SE) 0301476-8

(51) Int. Cl. C07D 333/32; C07D 209/82; C07D 209/44; C07C 201/02

(52) U.S. Cl. 548/444; 548/470; 549/64; 558/482

(57) ABSTRACT

The present invention relates to a new process for the preparation of NO-donating compounds using a sulfonated intermediate. The invention relates to new intermediates prepared therein suitable for large scale manufacturing of NO-donating compounds. The invention further relates to the use of the new intermediates for the manufacturing of pharmaceutically active NO-donating compounds. The invention further relates to a substantially crystalline form of NO-donating NSAIDs, especially 2-[2-(nitrooxy)ethoxy]ethyl {2-[(2,6-dichlorophenyl)amino]phenyl}acetate, the preparation thereof and to pharmaceutical formulations containing said crystalline form and to the use of said crystalline form in the preparation of a medicament.

### FIELD OF THE INVENTION

The present invention relates to a new process for the preparation of NO-donating compoundsi.e. compounds releasing nitrogen oxide, using a sulfonated intermediate. The invention relates to new intermediates prepared therein suitable for large scale manufacturing of NO-donating compounds. The invention further relates to the use of the new intermediates for the manufacturing of pharmaceutically active NO-donating compounds.

The invention further relates to a substantially crystalline form of NO-donating NSAIDs, especially 2-[2-(nitrooxy)ethoxy]ethyl {2-[(2,6-dichlorophenyl)amino]phenyl}acetate, the preparation thereof and to pharmaceutical formulations containing said crystalline form and to the use of said crystalline form in the preparation of a medicament.

### BACKGROUND TO THE INVENTION

NO donating compounds are compounds having a NO or NO₂ group linked to the pharmaceutically active compound. A linker may be used between the pharmaceutically active compound and the NO or NO₂ group.

The advantage of NO donating compounds compared to the parent compound are among others a good tolerance and the reduction of gastrointestinal side effects. This is especially true for NO donating analogues of NSAIDs such as diclofenac and ketoprofen.

NO donating analogues of NSAIDs are known for their pharmaceutical activity as antiinflammation and/or analgesic agents.

Different processes for the preparation of NO donating compounds have been described in the prior art.

In Cainelli, et al. (Tetrahedron Lett., 1985, 28, 3369-3372) and Cainelli, et al. (Tetrahedron 1985, 41, 1385-1392), the substitution of sulfonate esters with tetrabutylammonium nitrate or an ion-exchanger with nitrate ions in a solvent such as pentane, toluene or benzene, is described. During this process high temperatures are used, which makes the process unsafe to use for large scale production.

Cainelli, et al. (J. Chem. Soc. Perkin Trans. I, 1987, 2637-2642) describe the nitrate substitution of sulfonate esters by reacting alkylmethanesulfonates with tetrabutylammonium nitrate in toluene.

In Kawamura, et al. (Chem. Parm. Bull., 1990, 38, 2092-2096) an alkylphenylsulfonate is reacted with tetrabutylammonium nitrate in toluene.

The costs for the tetraalkylammonium nitrate sources used in stoichiometric amounts as described in these prior art documents are economically undesirable for large-scale manufacturing of NO donating compounds. Processes wherein cheaper and low molecular weight alkali metal nitrates may be used are preferred for economical reasons. However, tetraalkylammonium nitrates may be used as phase transfer catalysts in substoichiometric amounts.

In Hwu, et al. (Synthesis, 1994, 471-474) the preparation of nitrate esters from sulfonic acid esters is described. The rather high temperatures and long reaction times used in combination with the low stability of the end products obtained, makes this process less suitable for large-scale production. In addition, the molar excess of sodium nitrate is at least twice as large as in the present invention, which increases costs and may give more waste problems. Further, the crude product obtained by the method according to Hwu et al, needs to be purified either by way of chromatography or distillation to obtain a pharmaceutically acceptable purity. Neither of these two purification options are appreciated for the large scale manufacturing of compounds.

ES 2,073,995 discloses the syntheses of alkyl nitrate esters from alkylsulfonates or 4-toluenesulfonates and metal nitrates using solvents such as dimethyl formamide, dimethyl acetamide, acetonitrile or dimethylsulfoxide. Using dimethyl acetamide or dimethylsulfoxide as solvent in the synthesis of NO donating compounds starting from for instance sulfonated intermediates gives a crude product which needs to be purified either by chromatography or by distillation to achieve a pharmaceutically acceptable purity.

Examples of NSAIDs are diclofenac (compound of formula Ia) and ketoprofen (compound of formula Id):

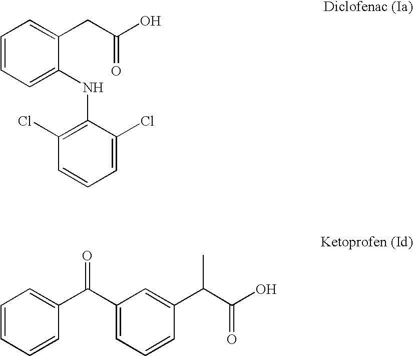

WO 94/04484 and WO 94/12463 disclose processes for the preparation of NO donating analogues of diclofenac and ketoprofen, respectively. In said processes a dihalide derivates is reacted with a salt of the carboxylic acid in DMF. The reaction products are converted into the final products by reaction with AgNO₃ in acetonitrile, in accordance with literature reports.

The process of the invention uses a sulfonated intermediate. This intermediate may be easily manufactured and is highly reactive for reactions with nitrate ions to form the corresponding nitrooxyalkyl ester.

Thus, there is a need for a more convenient and more economically efficient process for the manufacturing of large scale quantities of pharmaceutical quality of NO donating compounds, and their sulfonated intermediates, where factors like costs, manufacturing time, use of more environmentally friendly solvents, etcetera are vital for commercial application. The present invention provides for such a process.

In the formulation of drug compositions, it is important for the compound to be in a form in which it can be conveniently handled and processed. This is of importance for obtaining a commercially viable manufacturing process and for the manufacture of pharmaceutical formulations comprising the active compound.

Further, in the manufacture of drug compositions, it is important that a reliable, reproducible and constant plasma concentration profile of the compound is provided following administration to a patient.

Chemical stability and physical stability of the compounds are important factors. The compound, and formulations containing it, should be capable of being effectively stored over appreciable periods of time, without exhibiting a significant change in the active compound's physico-chemical characteristics such as its chemical composition, density, hygroscopicity and solubility.

Moreover, it is important to be able to provide the compound in a form, which is as chemically pure as possible.

Amorphous materials may present significant problems in this regard. Such materials are difficult to handle and to formulate, provide for unreliable solubility, and are often found to be unstable and chemically impure.

Thus, in the manufacture of commercially viable and pharmaceutically acceptable formulations, it is important, wherever possible, to provide a drug in a substantially crystalline and stable form.

It is to be noted, however, that this goal is not always achievable. Indeed, typically, it is not possible to predict, from molecular structure alone, what the crystallisation behaviour of a compound will be. This can usually only be determined experimentally.

The inventors have found that 2-[2-(nitrooxy)ethoxy]ethyl {2-[(2,6-dichlorophenyl)amino]phenyl}-acetate (compound IVa) can be obtained in a form that is both substantially crystalline and stable.

### DETAILED DESCRIPTION OF THE INVENTION

The present invention provides for a new process to prepare NO-donating compounds. Further, it provides for new intermediates and a process to prepare said intermediates, especially with regard to large-scale manufacturing.

The new manufacture process of NO-donating compounds is described below.

One embodiment of the invention relates to a process for the manufacturing of NO-donating compounds comprising; comprising; step 1, 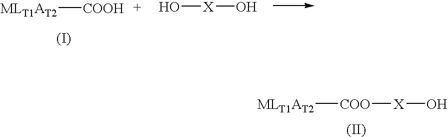
using an acidic or dehydrating agent and a solvent, optionally followed by purification using extraction or crystallisation, and step 2, 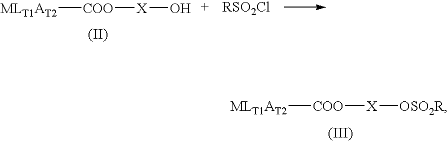
using a solvent, a base and optionally a catalyst, followed by purification using extraction and crystallisation, and step 3, 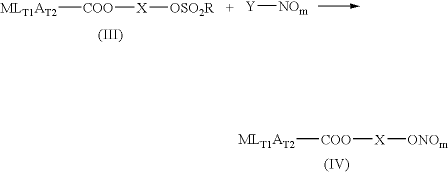
using a solvent and optionally a catalyst,
optionally followed by a crystallisation process for obtaining the compound of formula IV in a substantially crystalline form, and
   wherein:
M is a radical of a physiologically active compound;
L is O, S, (CO)O, (CO)NH, (CO)NR¹, NH, NR¹, wherein R¹ is a linear or branched alkyl group, or 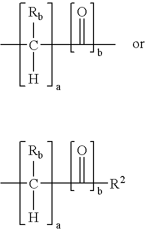
   wherein R_{b} is H, C₁₋₁₂alkyl or C₂₋₁₂alkenyl;
   R² is (CO)NH, (CO)NR¹, (CO)O, or CR¹ and a and b are independently 0 or 1;
A is a substituted or unsubstituted straight or branched alkyl chain;
X is a carbon linker;
R is selected from the group consisting of C₁-C₈ alkyl, phenyl, phenylmethyl,
C₁-C₄ alkylphenyl, halophenyl, nitrophenyl, acetylaminophenyl, halogen, CF₃ and n-C₄F₉; Y—NO₃ is lithium nitrate, sodium nitrate, potassium nitrate, magnesium nitrate, calcium nitrate, iron nitrate, zinc nitrate or tetraalkylammonium nitrate (wherein alkyl is a C₁-C₁₈-alkyl, which may be straight or branched);
m is 1 or 2; and
T1 and T2 are each independently 0, 1, 2 or 3;
with the proviso that
when ML_{TI}A_{T2}-COOH is naproxen then X is not (CH₂)₄.

Another embodiment of the invention relates to a process for the preparation of intermediates of formula III, which may be used for the manufacturing of NO-donating compounds comprising; step 1, 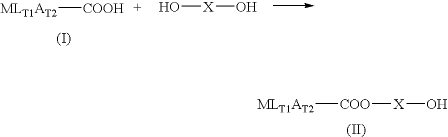
using an acidic or dehydrating agent and a solvent, optionally followed by purification using extraction or crystallisation, and step 2, 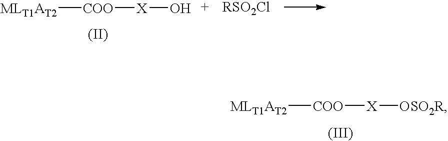
using a solvent, a base and optionally a catalyst, followed by purification using extraction and crystallisation, and
wherein M, L, A, T1, T2, X and R are as defined above.

The term “C₁-C₈ alkyl” means an alkyl having 1 to 8 carbon atoms and includes both straight and branched chain alkyl groups such as methyl, ethyl, n-propyl, i-propyl, n-butyl, i-butyl, t-butyl, etc.

The term “C₁-C₄ alkylphenyl” means methylphenyl, ethylphenyl n-propylphenyl, i-propylphenyl, n-butylphenyl, i-butylphenyl and t-butylphenyl.

The term “phenylmethyl” means benzyl.

The term “halo” and “halogen” refer to fluoro, chloro or bromo.

The term “halophenyl”, “nitrophenyl” and “acetylaminophenyl” refer to phenyl groups substituted with one or more halogen, nitro or acetylamino group.

The term “large scale” means a manufacturing scale in the range of “kilogram to multiton”.

M may be any radical of any physiologically active compound.

ML_{T1}A_{T2}-COOH may be any physiologically active carboxylic acid.

In one embodiment of the invention the group M is part of the molecule of an NSAID, COX 1 or COX 2 inhibitor.

In another embodiment of the invention the group M is selected from the group consisting of 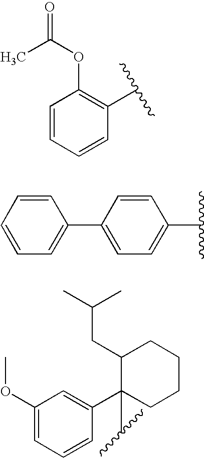 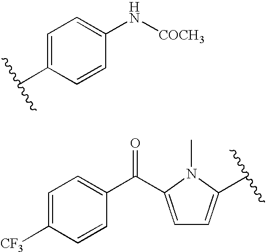
as decribed in WO 00/51988, and 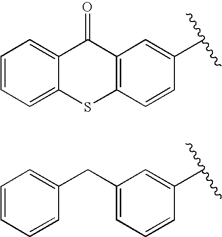
as described in U.S. Pat. No. 3,641,127, and 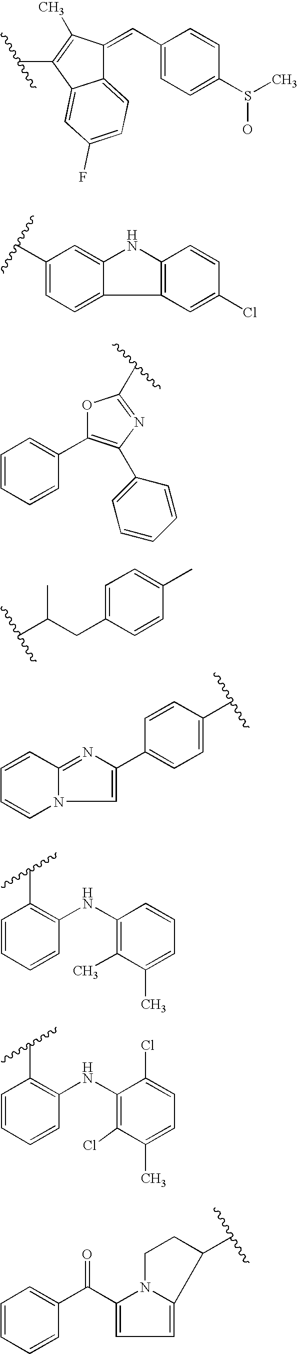
as described in WO 96/32946, and cycloalkyls as described in WO 98/25918 such as 2,2-dimethyl-cyclopropane-1-methanol, and 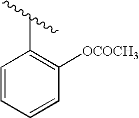
as described in CN 1144092, and or 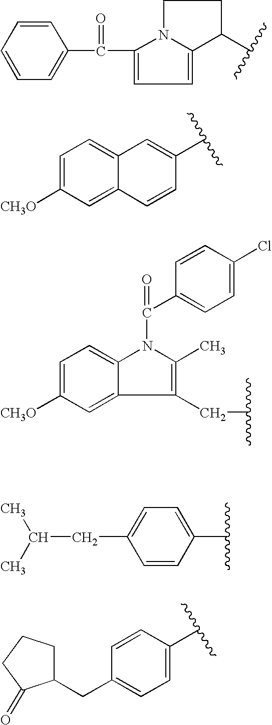
as described in WO 95/09831, and 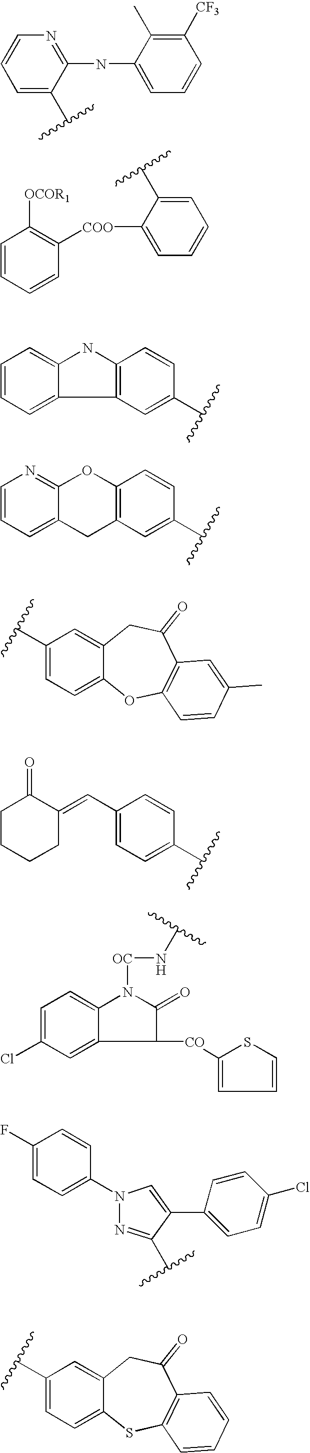 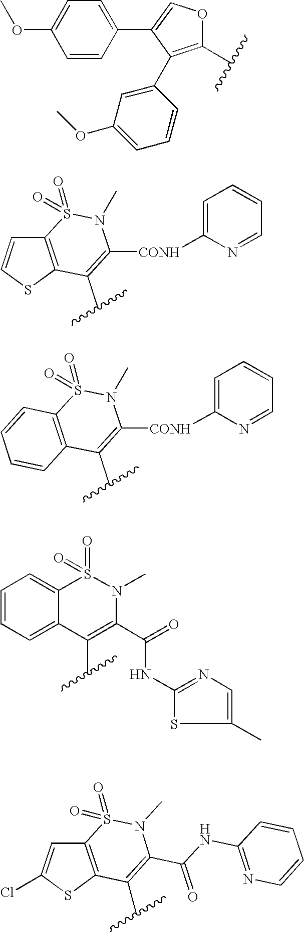
as decribed in WO 95/30641, and 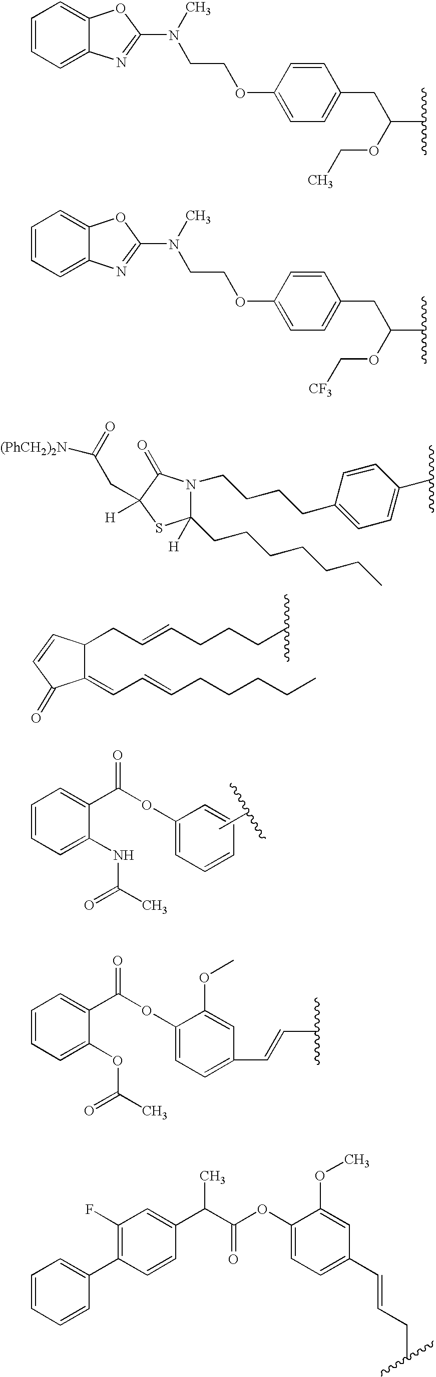 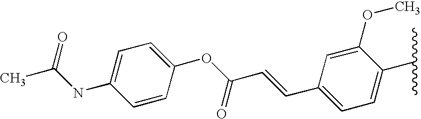 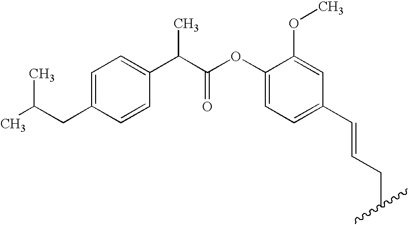
as described in WO 02/30866, and 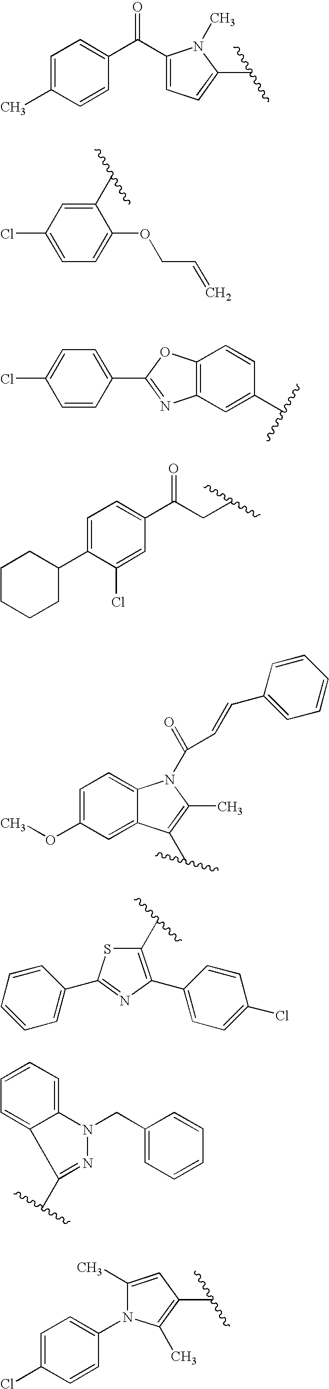 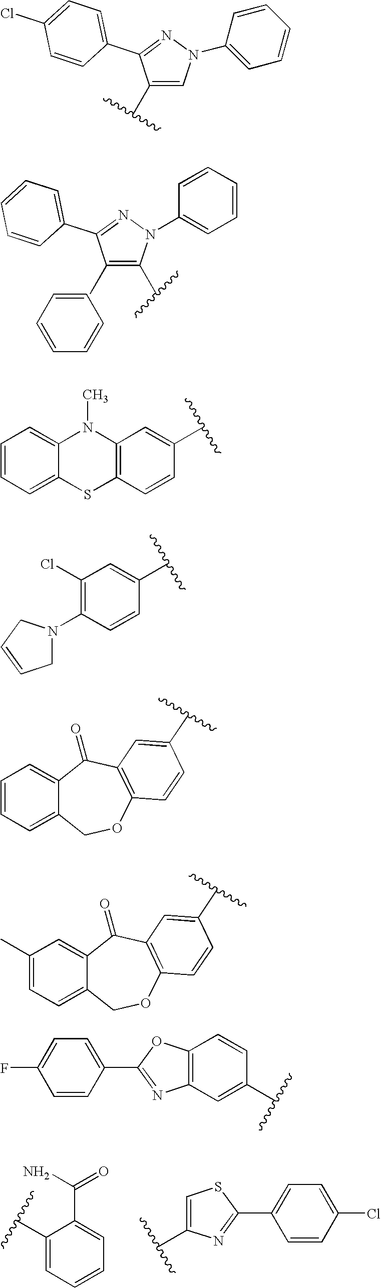 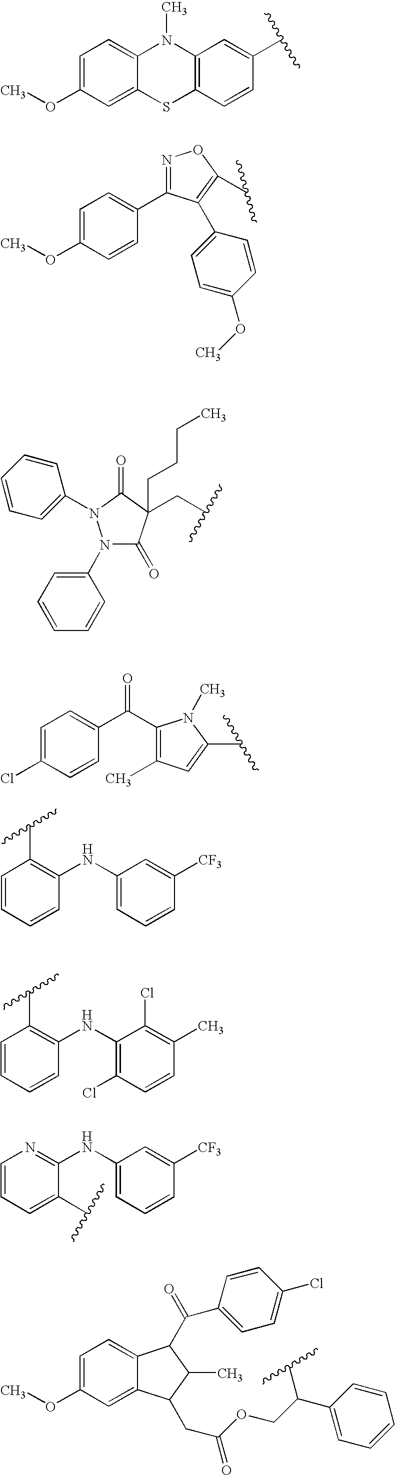 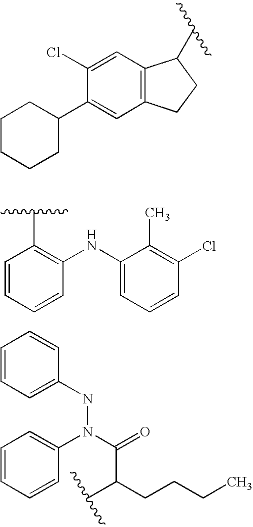
as described in U.S. Pat. No. 6,297,260.

In one embodiment of the invention L is selected from the group consisting of O, S, NH, NR¹, wherein R¹ is a linear or branched alkyl group, as described in WO 95/09831, and (CO) or (CO)O as described in WO 95/30641, and 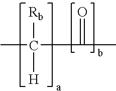
wherein R_{b} is H, C₁₋₁₂alkyl or C₂₋₁₂alkenyl and a and b are independently 0 or 1, as described in WO 02/053188, and 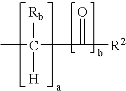
wherein R_{b}, a and b are defined as above; and R² is (CO)NH, (CO)NR¹, (CO)O, or CR¹.

In another embodiment of the invention A is selected from the group consisting of —(CH₂)ₙ—, whereby n is 0, 1, 2, 3 or 4, 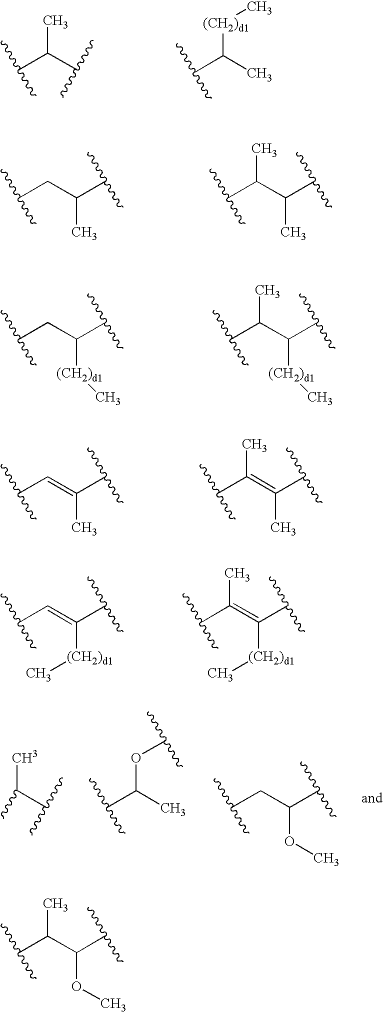
wherein d1 is 1, 2 or 3.

In a further embodiment of the invention A is selected from the group consisting of 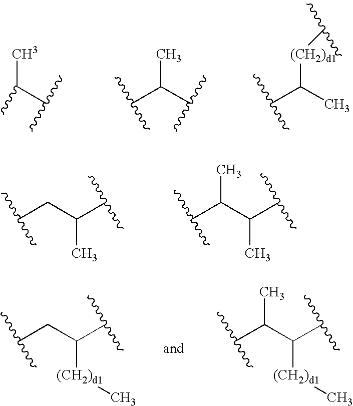
wherein d1 is 1, 2 or 3.

The linker carbon X may be selected from the group consisting of 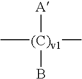
wherein A′ and B are chosen among hydrogen, linear or branched or cyclic substituted or non substituted alkyl group, and v1 is comprised between 1 and 10 as described in WO 95/09831, and —(CH₂—CH₂—O)₂—, or a cycloalkyl having 5 to 7 carbon atoms optionally substituted, and 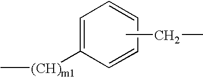
wherein m1 is comprised between 0 and 3, and 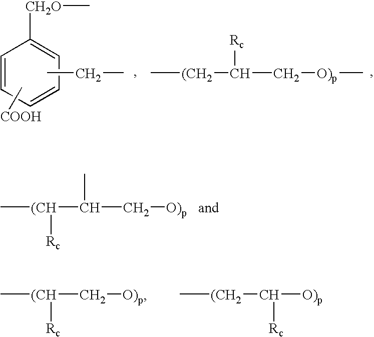
wherein R_{c} is H or methyl, and
p is comprised between 0 and 6, as described in WO 95/30641 and WO 02/92072, and —(CH₂)_{q}—OCO—(CH₂)ᵣ, wherein q and r each independently comprise between 0 and 6, and 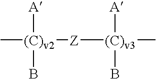
wherein Z is O, SO, S or a saturated, unsaturated or aromatic 5 or 6 membered ring or 5 or 6 membered heterocyclic ring containing one or more heteroatoms selected independently from N, O and S,
wherein said ring may optionally be substituted, and v2 and v3 are independently comprised between 0 and 4 and

In one embodiment of the invention X is selected from the group consisting of linear, branched or cyclic —(CH₂)—_{w1} wherein w1 is an integer of from 2 to 10; —(CH₂)_{w2}—O—(CH₂)_{w3}— wherein w2 and w3 are integers of from 2 to 10; and —CH₂—C₆H₄—CH₂—.

In another embodiment of the invention X is selected from the group consisting of linear —(CH₂)_{w1}— wherein w1 is an integer of from 2 to 6; —(CH₂)₂—O—(CH₂)₂— and —CH₂—C₆H₄—CH₂—.

In a further embodiment of the invention R is selected from the group consisting of C₁-C₈ alkyl, phenyl, phenylmethyl, C₁-C₄ alkylphenyl, halophenyl, nitrophenyl, acetylaminophenyl and halogen.

In one embodiment of the invention the group ML_{T1}A_{T2} is selected from the group consisting of

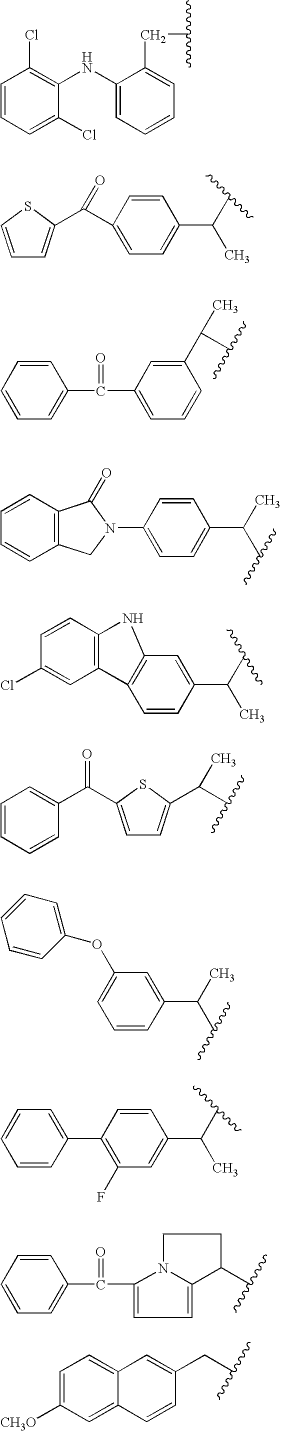

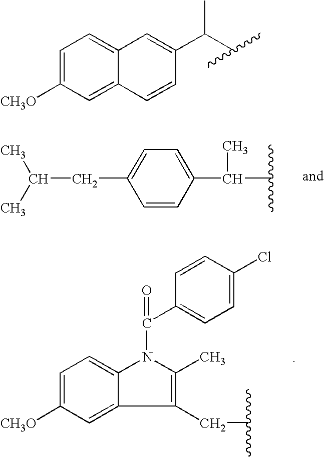

In another embodiment of the invention the group ML_{T1}A_{T2} is selected from the group consisting of

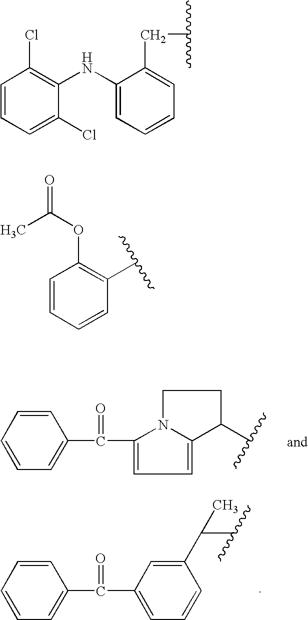

In a particular embodiment the group ML_{T1}A_{T2} is 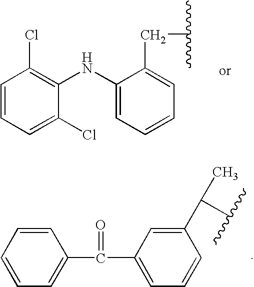
The Process in Detail

Step 1 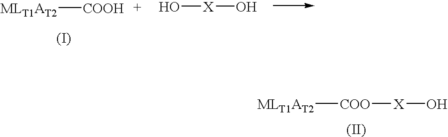
wherein M, L, A, T1, T2 and X are as defined above.

ML_{T1}A_{T2}-COOH may be esterified in reaction step 1 by using acid catalysed esterification in the presence of diethylene glycol as described in DE 88-3811118 where p-toluenesulfonic acid is used.

The esterification step 1 may be performed in a manner known to a person skilled in the art, for example by treating the compound of formula I, for example diclofenac and diethylene glycol with an acidic or dehydrating agent.

One embodiment relates to the process of the invention whereby an acidic or dehydrating agent in step 1 is selected from the group consisting of sulphuric acid or its salts, perchloric acid (e.g. 70%) or other suitable acids such as polystyrene sulphonic acids, zeolites, acidic clays, sand in combination with strong hydrophilic acids such as perchloric acid or gaseous hydrogen chloride and montmorillonites.

Compounds of formula II may also be prepared in the same manner using 1,4-butanediol, 1,3-propanediol and triethyleneglycol respectively. In ES 85-548226 thionyl chloride is used to catalyse the esterification.

The acids may be used in the gas, fluid or solid form. The solid heterogeneous acids can relatively easily be filtered from the reaction solution and re-used in large-scale production processes.

Examples of other coupling reagents useful for the esterification step 1 are carbodiimides such as N,N′-dicyclohexylcarbodiimide (DCC), acid chlorides such as oxalyl chloride, chloroformates such as isobutyl chloroformate or other reagents such as cyanuric chloride, N,N′-carbonyldiimidazole, diethyl chlorophosphite, 2-chloro-1-methyl-pyridinium iodide and 2,2′-dipyridyl disulphide.

One embodiment relates to the process of the invention whereby the solvent in step 1 is a non-polar and/or non acidic solvent.

The reaction step 1 may be performed in a solvent selected from the group comprising of aromatic hydrocarbons such as benzene or toluene, aliphatic hydrocarbons such as n-heptane, ketones such as methyl isobutylketone, ethers such as tetrahydrofuran or diethyleneglycol dimethyl ether and chlorinated hydrocarbons such as dichloromethane or chlorobenzene, or mixtures thereof.

Alternatively, an excess of the corresponding diol may be used as solvent optionally mixed with any of the other organic solvents mentioned above.

Compounds of formula II as obtained in step 1 may be purified by way of extraction, batch-wise or continuously, to obtain a solution comprising the compound of formula II having a chromatographic purity of at least 92% and preferably more than 97% (after extraxtion step i) and an alkylene diol or alkylene glycol content below about 0.5% (w/w) (after extraction step ii).

Extraction Step i)

In this extraction step the chromatographic purity is improved. The solution used in this extraction step may comprise a mixture of i) alkylene diol or alkylene glycol, ii) water and/or a low molecular weight aliphatic alcohol and iii) a hydrocarbon solvent or mixtures thereof or mixtures of organic solvents with hydrocarbon solvents.

The low molecular weight aliphatic alcohols may be selected from the group consisting of methanol, ethanol and propanol, or mixtures thereof.

The hydrocarbon solvents used for extraction step i) may be selected from the group comprising of toluene, cumene, xylenes, ligroin, petroleum ether, halobenzenes, heptanes, hexanes, octanes, cyclohexanes, cycloheptanes, and the like, or mixtures thereof.

Suitable organic solvents used for extraction step i) may be selected from the group comprising of ketones such as methyl iso-butyl ketone, ethers such as di-n-butyl ether or tert-butyl methyl ether and aliphatic esters such as ethyl acetate or n-butyl acetate and haloalkanes such as dichloromethane, or mixtures thereof.

The purified compound of formula II is obtained as a solution in a mixture of alkylene diol or alkylene glycol with water and/or a low molecular weight aliphatic alcohol.

Extraction Step ii)

This extraction is performed to lower the alkylene diol or alkylene glycol-content and performed after extraction step i) wherein the chromatographic purity is improved as described above. The solution may comprise i) a mixture of water and/or a low molecular weight aliphatic alcohol and ii) an organic solvent or mixtures of organic solvents.

The low molecular weight aliphatic alcohols may be selected from the group consisting of methanol, ethanol and propanol, or mixtures thereof.

A suitable organic solvent used for extraction step ii) may be selected from the group comprising of aromatic hydrocarbons such as toluene, cumene or xylenes, ketones such as methyl iso-butyl ketone, ethers such as di-n-butyl ether or tert-butyl methyl ether and aliphatic esters such as ethyl acetate or n-butyl acetate and haloalkanes such as dichloromethane, or mixtures thereof.

The total amount of solvents used in the esterification process step 1, may vary between 0 to 100 volume parts per weight of starting material.

The temperature of the esterification step 1 may be between −100° C. to +130° C., preferably between 0° C. and +120° C.

Step 2 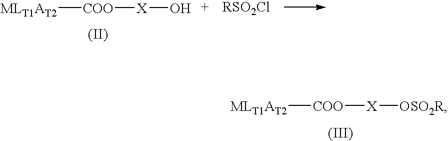
wherein:
M, L, A, T1, T2, X and R are as defined above.

The reaction condition in step 2 would suitably involve an excess of RSO₂Cl in an organic solvent or a mixture of organic solvents.

A suitable solvent in step 2 may be selected from the group comprising of aromatic hydrocarbons such as toluene, cumene or xylenes, ketones such as methyl iso-butyl ketone, ethers such as di-n-butyl ether, tert-butyl methyl ether or tetrahydrofuran, aliphatic nitriles such as acetonitrile and aliphatic esters such as ethyl acetate or n-butyl acetate and haloalkanes such as dichloromethane, or mixtures thereof.

One embodiment relates to the process of the invention whereby the solvents in step 2 are selected from a group consisting of toluene, cumene, xylenes, ethyl acetate, acetonitrile, butyl acetate and isopropyl acetate.

A base may be added in step 2. In one embodiment of the invention the base in step 2 may be selected from the group consisting of triethylamine, pyridine, N-methylmorpholine, diisopropylethylamine, tributylamine and N-methyl-piperidine.

Another embodiment relates to the process of the invention whereby the base in step 2 is triethylamine or N-methylmorpholine.

A further embodiment relates to the process of the invention whereby a catalyst such as 4-(dimethylamino)pyridine may optionally be used in step 2.

Compounds of formula III as obtained in step 2 may be purified by crystallisation from an organic solvent to obtain a crystalline solid having a chemical purity of about 95% and particularly about 98%.

Another embodiment relates to the process of the invention whereby an antisolvent is used in the crystallization of compound of formula III in step 2.

In a further embodiment of the invention the solvent used for the crystallisation may be selected from the group comprising of aromatic hydrocarbons such as toluene, cumene or xylenes, ketones such as methyl iso-butyl ketone, ethers such as di-n-butyl ether, tert-butyl methyl ether or tetrahydrofuran, aliphatic nitrites such as acetonitrile and aliphatic esters such as ethyl acetate or butyl acetate, or mixtures thereof.

Yet another embodiment relates to the process of the invention whereby the solvent used for the crystallisation in step 2 is selected from the group consisting of toluene, cumene, xylenes, ethyl acetate, acetonitrile, butyl acetate and isopropyl acetate, or mixtures thereof.

Yet a further embodiment relates to the process of the invention whereby the antisolvent used for the crystallisation in step 2 is selected from the group comprising of ligroin, petroleum ether, halobenzenes, heptanes, hexanes, octanes such as isooctane, cyclohexanes, cycloheptanes and alcohols, or mixtures thereof.

Step 3 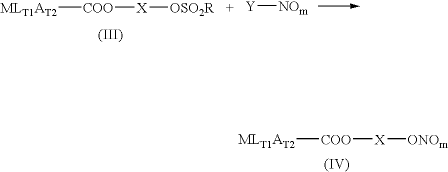
wherein M, L, A, T1, T2, X, R, m and Y are as defined above.

In step 3 of the manufacturing process, a compound of formula IV is obtained by reacting the compound of formula III with a nitrate source (Y—NO₃) optionally in the presence of a solvent.

This reaction may be performed with a nitrate source Y—NO₃ selected from the group consisting of lithium nitrate, sodium nitrate, potassium nitrate, magnesium nitrate, calcium nitrate, iron nitrate, zinc nitrate and tetraalkylammonium nitrate (wherein alkyl is a C₁-C₁₈-alkyl, which may be straight or branched).

One embodiment relates to the process of the invention whereby the nitrate sources Y—NO3 in step 3 is selected from the group consisting of lithium nitrate, sodium nitrate, potassium nitrate, magnesium nitrate and calcium nitrate, or mixtures thereof.

Another embodiment relates to the process of the invention whereby the organic solvent in step 3 is a polar aprotic solvent.

In a further embodiment of the invention the polar aprotic solvents used in step 3 may be selected from the group comprising of N-methylpyrrolidinone, N,N-dimethylacetamide, sulpholane, tetramethylurea, 1,3-dimethyl-2-imidazolidinone and nitriles such as acetonitrile, or mixtures thereof.

Other solvents may be aromatic hydrocarbons such as toluene, aliphatic hydrocarbons such as n-heptane, ketones such as methyl ethyl ketone, methyl isobutylketone, ethers such as tetrahydrofuran or diethyleneglycol dimethyl ether, chlorinated hydrocarbons such as chlorobenzene, aliphatic esters such as ethyl acetate, butyl acetate or isopropyl acetate, nitrated hydrocarbons such as nitromethane, ethylene glycols such as polyethylene glycol and mixtures of these, optionally with an added aliphatic alcohols such as methanol, ethanol, n-propanol, i-propanol, n-butanol, i-butanol or t-butanol.

One embodiment of the invention relates to the process of the invention whereby the organic solvent in step 3 is selected from the group consisting of N-methylpyrrolidinone, sulpholane, tetramethylurea, 1,3-dimethyl-2-imidazolidinone, acetonitrile, methyl isobutylketone, ethyl acetate, butyl acetate and isopropyl acetate, or mixtures thereof.

The nitration step 3 may also be performed in water, optionally in combination with any of the above listed organic solvents.

The nitration step 3 may optionally be performed in the presence of a phase-transfer-catalyst.

One embodiment relates to the process of the invention whereby the phase transfer-catalyst in step 3 is selected from the group consisting of tetraalkylammonium salt, arylalkylammonium salt, tetraalkylphosphonium salt, arylalkylphosphonium salt, crown ether, pentaethylene glycol, hexaethylene glycol and polyethylene glycols, or mixtures thereof.

Crystallisation of Compounds of Formula IV

Compounds of formula IV as obtained in step 3 may be purified by crystallisation from an organic solvent optionally using hydrocarbons, alcohols or water as anti solvent to obtain a crystalline solid product of a chemical purity of 90% and particularly about 95%.

One embodiment relates to the process of the invention whereby the compound of formula IV in step 3 is extracted batch-wise or continuosly and crystallised from an organic solvent optionally using an anti solvent to obtain a crystalline solid having a chemical purity of at least 95%.

Preferably, the crystallisation is performed in an appropriate solvent system. Crystallisation may also be performed in the absence of a solvent system. Other examples of crystallisation include crystallisation from a melt, under supercritical conditions, or achieved by sublimation.

Crystallisation of compounds of formula IV from an appropriate solvent system may be achieved by attaining supersaturation in a solvent system, which comprises compound of formula IV. This may be done by cooling the solvent system, by evaporating the solvent, by adding a suitable antisolvent or by any combination of these methods. Crystallisation may also be affected by decreasing the solubility of the compound by the addition of a salt such as for example NaCl.

The crystallisation process may be started from the reaction solution comprising compound of formula IV as obtained after the preparation of said compound.

Also, the crystallisation process may be started from the dry compound of formula IV.

Alternatively, the crystallisation process may be started after extracting compound of formula IV from the reaction solution.

One embodiment of the invention relates to the process described above whereby the crystallisation process for compound of formula IV comprises the following steps:
a) i) dissolving the compound in a solvent; or,
   ii) extracting the compound from the reaction solution into a solvent; or,
   iii) starting from the reaction solution comprising said compound;
b) evaporating the solvent;
c) adding an anti-solvent and/or cooling
d) isolating the crystals formed, and optionally;
e) recrystallising the crystals formed in step c); or isolated in step d).

Another embodiment of the invention relates to the process described above whereby the crystallisation process for compound 2-[2-(nitrooxy)-ethoxy]ethyl {2-[(2,6-dichlorophenyl)amino]phenyl}acetate (IVa) comprises the following steps:
a) extracting the compound from the reaction solution into a solvent;
b) evaporating the solvent;
c) adding an anti-solvent and/or cooling
d) isolating the crystals formed, and optionally;
e) recrystallising the crystals formed in step c); or isolated in step d).

The substantially crystalline form of 2-[2-(nitrooxy)-ethoxy]ethyl {2-[(2,6-dichlorophenyl)amino]phenyl}acetate is hereinafter referred to as “Form A of compound IVa”.

A further embodiment of the invention, there is provided a process for the production of Form A of compound IVa which comprises crystallising 2-[2-(nitrooxy)ethoxy]ethyl {2-[(2,6-dichlorophenyl)amino]phenyl}acetate.

Suitable solvents used for the crystallisation process may be selected from the group comprising of lower alkyl acetates e.g. linear or branched C₁₋₆ alkyl acetates such as ethyl acetate, iso-propyl acetate or butyl acetate, lower linear or branched C₂₋₆ alkyl alcohols, preferably C₂₋₄ alkyl alcohols such as ethanol or iso-propanol, aliphatic and aromatic hydrocarbons e.g. C₅₋₁₂ aliphatic hydrocarbons or C₆₋₁₀ aromatic hydrocarbons such as isooctane, cumene, xylenes, n-heptane, 1-methyl-2-pyrrolidinone or toluene, dialkyl ketones e.g. di-C₁₋₆ alkyl ketones such as acetone, methyl ethyl ketone, methyl iso-butyl ketone or 4-methyl-2-pentanone, dialkyl ethers e.g. di-C₁₋₆ alkyl ethers such as di-iso-propyl ether, di-n-butyl ether, tert-butyl methyleter or tetrahydrofuran, aliphatic nitriles such as acetonitrile and water, or mixtures thereof.

One embodiment of the invention relates to the crystallisation process described above whereby the solvent in step a) is selected from the group comprising of lower alkyl acetates, lower alkyl alcohols, aliphatic hydrocarbons, aromatic hydrocarbons, heteroaromatic hydrocarbons, dialkyl ketones, dialkyl ethers, nitriles and water, or mixtures thereof.

Another embodiment of the invention relates to the crystallisation process described above whereby the solvent in step a) is selected from the group consisting of ethyl acetate, iso-propyl acetate, butyl acetate, ethanol, iso-propanol, isooctane, n-heptane, toluene, 1-methyl-2-pyrrolidinone, methyl ethyl ketone, methyl iso-butyl ketone, di-iso-propyl ether, tert-butyl methylether, acetonitrile and water, or mixtures thereof.

A further embodiment relates to the crystallisation process described above whereby the solvent is selected from the group consisting of butylacetate, isopropanol, isooctane, acetone, acetonitrile and water, or mixtures thereof.

Solvents may also be employed as “antisolvents” (i.e. a solvent in which a compound is poorly soluble), and may thus aid the crystallisation process.

In one embodiment of the invention the antisolvent in step b) of the crystallisation process is selected from the group comprising of ethanol or 2-propanol, toluene, cumene, xylenes, ligroin, petroleum ether, halobenzenes, heptanes, hexanes, octanes, cyclohexanes and cycloheptanes, or mixtures thereof.

Further purification of the compound may be affected by recrystallisation and/or slurrying. The recrystallisation may be done from an appropriate solvent system for example linear or branched alkyl acetates such as ethyl acetate, iso-propyl acetate and butyl acetate, ketones such as acetone and 4-methyl-2-pentanone, aromatic hydrocarbons such as toluene and 1-methyl-2-pyrrolidinone, which may include an antisolvent for example water or a lower alkyl alcohols such as ethanol and iso-propanol or aliphatic hydrocarbons such as isooctane and n-heptane, or a combination of these solvents.

A further embodiment of the invention relates to the crystallisation process described above whereby the solvent in step d) is selected from the group comprising of aromatic hydrocarbons such as toluene, cumene or xylenes, ketones such as methyl iso-butyl ketone, ethers such as di-n-butyl ether, tert-butyl methyl ether or tetrahydrofuran, aliphatic nitriles such as acetonitrile and aliphatic esters such as ethyl acetate or n-butyl acetate and haloalkanes such as dichloromethane, or mixtures thereof, optionally together with an antisolvent selected from the group consisting of water, ethanol, iso-propanol, isooctane and n-heptane, or mixtures thereof.

Yet another embodiment of the invention relates to the crystallisation process described above whereby the solvent in step d) is selected from the group consisting of toluene, cumene, xylenes, methyl iso-butyl ketone, di-n-butyl ether, tert-butyl methyl ether, tetrahydrofuran, acetonitrile, n-butyl acetate and dichloromethane, or mixtures thereof, optionally together with an antisolvent selected from the group consisting of water, ethanol, iso-propanol, isooctane and n-heptane, or mixtures thereof.

Compounds of formula IV may for the recrystallisation, for example, first be dissolved in an organic solvent such as acetone and then washed with an antisolvent such as water, followed by cooling and filtering of the crystals obtained. After filtering the crystals may be further washed with a liquid, whereafter the liquid may be evaporated and the crystals dried.

Crystal forms of compounds of formula IV may be isolated using conventional techniques such as decanting, filtering or centrifuging.

The invention relates to a compound of compound IV obtainable by the processes as described above.

One embodiment of the invention relates to Form A of compound IVa crystallised according to the processes described above, whereby the chemical purity of Form A of compound IVa is above 95%, preferably above 98%, more preferably above 99%.

When Form A of compound IVa is crystallised, and/or recrystallised, as described herein, the resultant crystal, is expected to have improved chemical, physical and solid state stability.

According to one embodiment of the invention there is provided 2-[2-(nitrooxy)ethoxy]-ethyl {2-[(2,6-dichlorophenyl)amino]phenyl}acetate (IVa) in a substantially crystalline form.

Another embodiment of the invention relates to the anhydrate form of compound IVa. The preparation and characterisation of the anhydrate form are described hereinafter.

Although we have found that it is possible to produce 2-[2-(nitrooxy)ethoxy]ethyl {2-[(2,6-dichlorophenyl) amino]phenyl}acetate in a form which is more than 90% crystalline, by “substantially crystalline” we include greater than 50%, preferably greater than 60%, and more preferably greater than 70% crystalline.

The “degree (%) of crystallinity” may be determined using X-ray powder diffraction (XRPD). Other techniques, such a solid state NMR, FT-IR, Raman spectroscopy, differential scanning calorimetry (DSC) and microcalorimetry, may also be used as complementary methods.

One embodiment of the invention relates to Form A of compound IVa characterised by the major peaks in the X-ray powder diffractogram as shown in table 1 of Example 5a.

Form A of compound IVa may be characterised by its unit cell.

Another embodiment of the invention relates to Form A of compound IVa characterised by having a monoclinic unit cell with parameters a=13.79 Å, b=11.90 Å, c=13.01 Å, α=90°, β=94.0°, γ=90°.

Form A of compound IVa is expected to be chemically and physically stable for a prolonged period of time under storage conditions as defined below.

The term “stability” and “stable” as defined herein shall refer to chemical stability and physical stability.

The term “chemical stability” shall mean that Form A of compound IVa can be stored in an isolated solid form, or in the form of a solid formulation optionally in admixture with pharmaceutically acceptable carriers, diluents or adjuvants, under storage conditions, with an insignificant degree of chemical degradation or decomposition.

The term “physical stability” shall mean that Form A of compound IVa can be stored in an isolated solid form, or in the form of a solid formulation optionally in admixture with pharmaceutically acceptable carriers, diluents or adjuvants, under storage conditions, with an insignificant degree of physical degradation (e.g. crystallisation, recrystallisation, solid state phase transition, hydration, dehydration, solvatisation or desolvatisation).

Form A of compound IVa is expected to have improved chemical and physical characteristics such as improved solubility, thermal stability, light stability, hygroscopic stability, etcetera.

The invention relates also to the manufacturing of compounds of formula IVa, IVb, IVc and IVd. The diclofenac compounds a, b and c are distinguished from each other by the difference in linker X.

In the compounds of formula Ia, IIIa and IVa the linker X is C₂H₄OC₂H₄.

In the compounds of formula IIb, IIIb and IVb the linker X is C₄H₈.

In the compounds of formula IIc, IIIc and IVc the linker X is C₂H₄OC₂H₄OC₂H₄.

Compounds IId, IIId and IVd are ketoprofen compounds whereby the linker X is C₃H₆.

One embodiment of the invention relates to a process for the manufacturing of NO donating diclofenac of formula IVa, IVb or IVc, comprising:
step 1, reacting a compound of formula Ia with HO—X—OH, wherein X is C₂H₄OC₂H₄, C₄H₈ or C₂H₄OC₂H₄OC₂H₄, to obtain compounds of formula Ia, IIb or IIc, 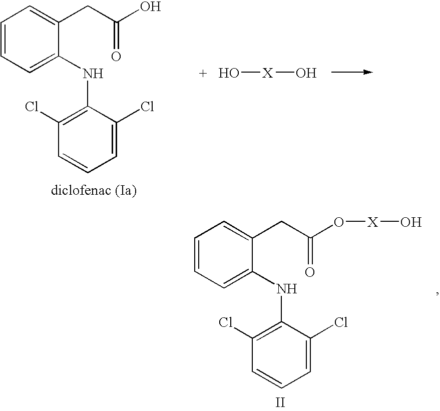
   followed by,
step 2, reacting the compounds of formula Ia, IIb or IIc with RSO₂Cl, wherein R is as defined above, to obtain compounds of formula IIIa, IIIb or IIIc, 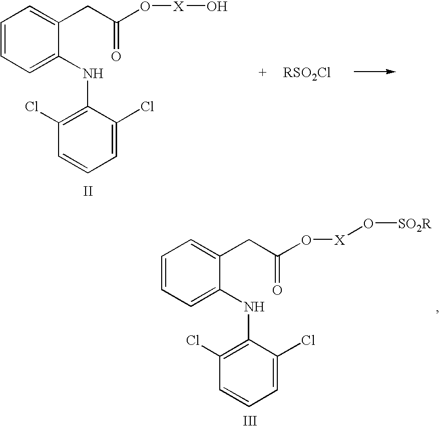
   followed by,
step 3, reacting the compounds of formula IIIa, IIIb or IIIc with a nitrate source Y—NO₃, wherein Y is as defined above, to obtain compounds of formula IVa, IVb or IVc, 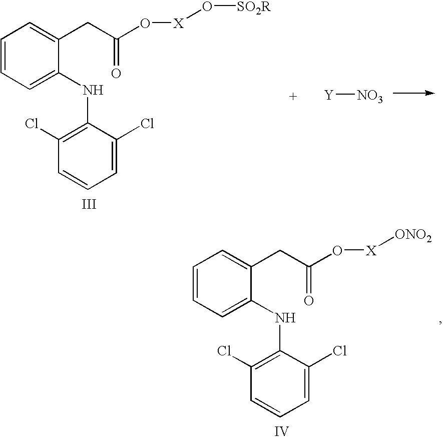
   followed by,
crystallising the compounds of formula IVa, IVb or IVc using the following steps:
   a) extracting the compound from the reaction solution into a solvent;
   b) evaporating the solvent;
   c) adding an anti-solvent and/or cooling
   d) isolating the crystals formed, and optionally;
   e) recrystallising the crystals formed in step c); or isolated in step d).

Another embodiment of the invention relates to a process for the manufacturing of NO donating diclofenac of formula IVa comprising:
step 1, reacting the compound of formula Ia with diethylene glycol to obtain a compound of formula IIa, 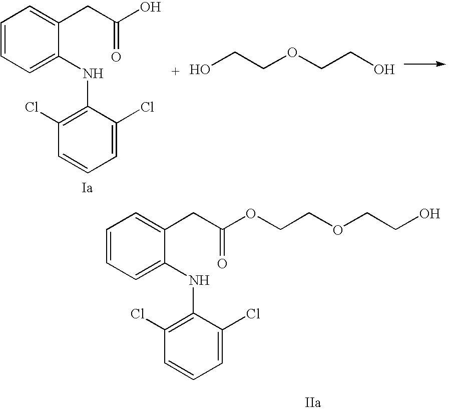
   followed by,
step 2, reacting the compound of formula IIa with RSO₂Cl, wherein R is as defined above, to obtain a compound of formula IIIa, 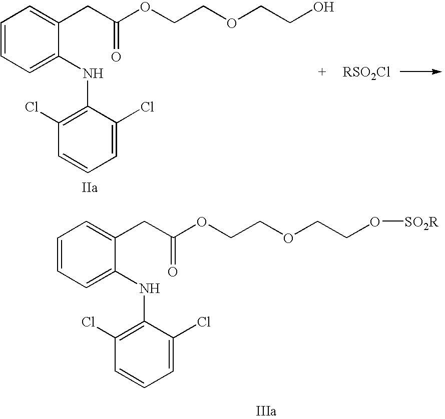
step 3, reacting the compound of formula IIIa with a nitrate source Y—NO₃, wherein Y is as defined above, to obtain a compound of formula IVa, 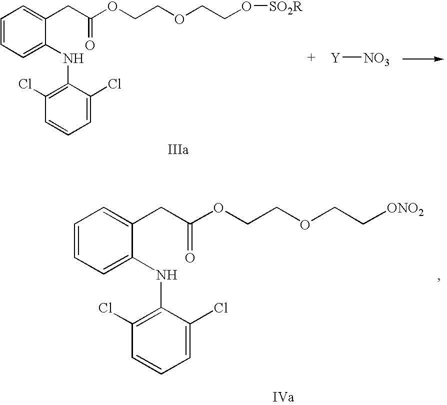
followed by crystallising the compound of formula IVa using the following steps:
   a) extracting the compound from the reaction solution into a solvent;
   b) evaporating the solvent;
   c) adding an anti-solvent and/or cooling
   d) isolating the crystals formed, and optionally;
   e) recrystallising the crystals formed in step c); or isolated in step d).

A further embodiment of the invention relates to a process for the manufacturing of NO donating ketoprofen of formula IVd comprising:
step 1, reacting a compound of formula Id with 1,3-propanediol to obtain a compound of formula IId, 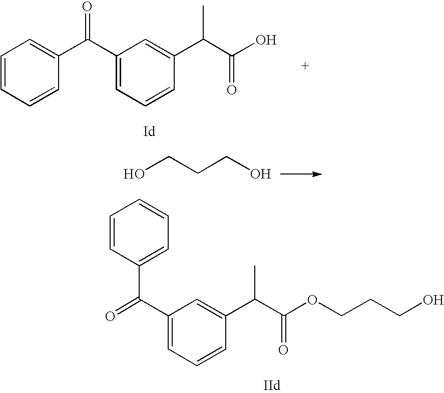
   followed by,
step 2, reacting the compound of formula IId with RSO₂Cl, wherein R is as defined above, to obtain a compound of formula IIId, 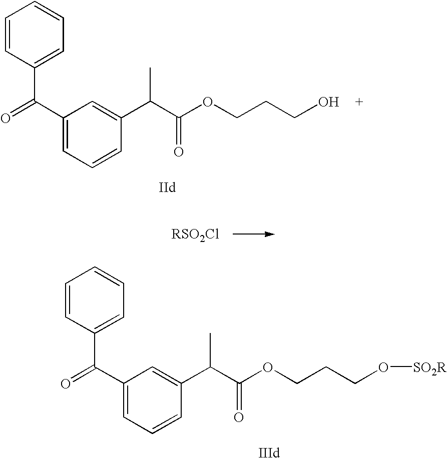
step 3, reacting the compound of formula IIId with a nitrate source Y—NO₃, wherein Y is as defined above, to obtain a compound of formula IVd,

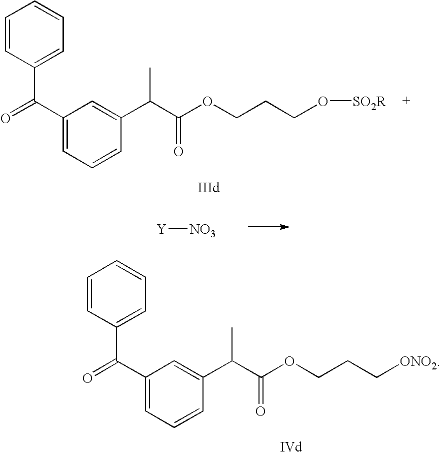

One embodiment of the invention relates to a process as described above for the manufacturing of the S-enantiomer of NO donating ketoprofen of formula IVd.

The temperature used in process step 1 and 2 may be between −100° C. and +130° C. The temperature is particularly kept below 130° C., because the stability of the end product might be affected by a high temperature. Reaction step 3 is particularly performed at a temperature below 90° C. The temperature used in the crystallization process may be below 0° C., for example down to −40° C.

One embodiment relates to the processes of the invention whereby the temperature is between −40° C. and 120° C.

Room temperature shall mean a temperature between 18° C. and 25° C.

The total amount of solvents may vary between 0 to 100 volume parts per weight of starting material.

Different reaction steps may need different reaction times.

In the processes of the invention the use of explosive intermediates such as nitrooxyalkanols are avoided. Furthermore, the new processes is commercially and environmentally more advantageous than the known processes.

Another advantage of the processes of the invention is that the enantiomeric purity of the starting material is at least maintained in the end products (IV) for which asymmetric carbons are present.

Intermediates

One embodiment of the invention relates to intermediates of formula III, ML_{T1}A_{T2}—X—O—SO₂R, wherein M, L, A, T1, T2, X and R are as defined above.

Another embodiment of the invention relates to compounds of formula IIIa, IIIb, IIIc and IIId: 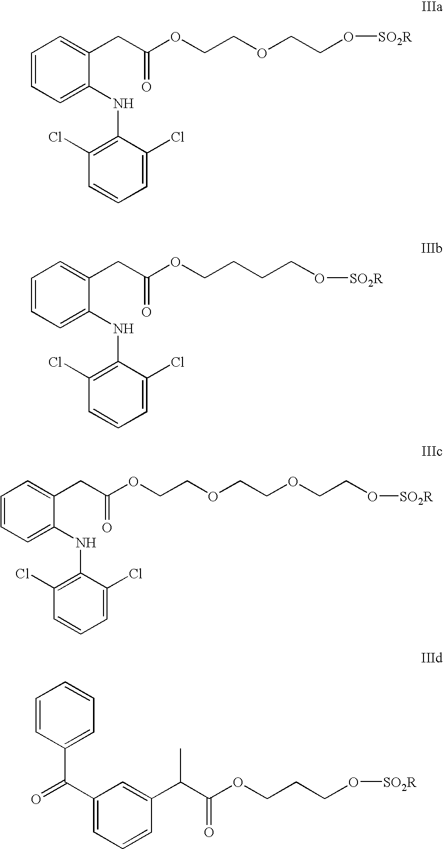
wherein R is selected from the group consisting of C₁-C₈ alkyl, phenyl, phenylmethyl, C₁-C₄ alkylphenyl, halophenyl, nitrophenyl, acetylaminophenyl, halogen, CF₃ and n-C₄F₉.

A further embodiment of the invention relates to the S-enantiomer of the compound of formula IIId 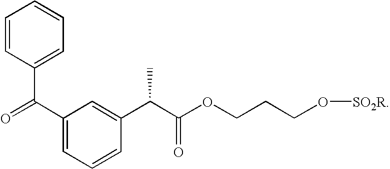
wherein R is selected from the group consisting of C₁-C₈ alkyl, phenyl, phenylmethyl, C₁-C₄ alkylphenyl, halophenyl, nitrophenyl, acetylaminophenyl, halogen, CF₃ and n-C₄F₉.

Yet another embodiment of the invention relates to compounds of formula IIIa, 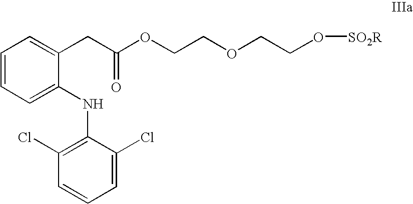
wherein R is selected from the group consisting of C₁-C8 alkyl, phenyl, phenylmethyl, C₁-C₄ alkylphenyl, halophenyl, nitrophenyl, acetylaminophenyl, halogen, CF₃ and n-C₄F₉.
Use

One embodiment of the invention relates to the use of the compounds of formula IIIa, IIIb, IIIc and IIId as defined above, as an intermediate for the manufacturing of 2-[2-(nitrooxy)ethoxy]ethyl {2-[(2,6-dichlorophenyl)amino]phenyl}acetate, 4-(nitrooxy)butyl {2-[(2,6-dichlorophenyl)amino]phenyl}acetate, 2-{2-[2-(nitrooxy)ethoxy]ethoxy}ethyl {2-[(2,6-dichlorophenyl)amino]phenyl}acetate, 3-(nitrooxy)propyl 2-(2-benzoylphenyl)-propanoate and 3-(nitrooxy)propyl (2S)-2-(2-benzoylphenyl)propanoate.

Another embodiment of the invention related to the use of the process as defined above for the large scale manufacturing of NO donating compounds of formula IV.

A further embodiment of the invention related to the use of the process as defined above for the large scale manufacturing of the compounds of formula IVa, IVb, IVc and IVd.

Medical Use

One embodiment of the invention relates to the use of the compounds of formula III, ML_{T1}A_{T2}—X—O—SO₂R, wherein M, L, A, T1, T2, X and R are as defined above, as an intermediate for the manufacturing of a pharmaceutically active compound.

Another embodiment of the invention relates to the use of intermediate compounds of formula IIIa, IIIb, IIIc and IIId as defined above, prepared according to the process described above under step 1 and 2, for the manufacturing of a medicament for the treatment of pain and/or inflammation.

A further embodiment of the invention relates to the use of Form A of compound IVa for the manufacturing of a medicament.

Form A of compound IVa can be used for the treatment of pain and/or inflammation.

Yet another embodiment of the invention relates to the use of Form A of compound IVa for the manufacturing of a medicament for the treatment of pain and/or inflammation.

Yet a further embodiment of the invention relates to a method of treatment of pain and/or inflammation, comprising administration to a patient in need of such treatment, a therapeutically effective amount of Form A of compound IVa.

Pharmaceutical Preparations

Compounds of formula IV will normally be administered orally, rectally or parenterally in a pharmaceutically acceptable dosage form. The dosage form may be solid, semisolid or liquid formulation. Usually, the active compound will constitute between 0.1 and 99% by weight of the dosage form, preferably between 0.5 and 20% by weight for a dosage form intended for injection and between 0.2 and 80% by weight for a dosage form intended for oral administration.

A pharmaceutical formulation comprising compounds of formula IV may be manufactured by conventional techniques.

Suitable daily doses of compounds of formula IV in therapeutical treatment of humans are about 0.001-100 mg/kg bodyweight for parenteral administrations and about 0.01-100 mg/kg bodyweight for other administration routes.

One embodiment of the invention provides a pharmaceutical formulation comprising as active compound, a therapeutically effective amount of Form A of compound IVa, optionally in association with diluents, excipients or carriers.

Another embodiment of the invention relates to a formulation comprising an aqueous solution containing Form A of compound IVa.

A further embodiment of the invention relates to a pharmaceutical formulation comprising Form A of compound IVa, optionally in association with diluents, excipients or carriers.

Yet another embodiment of the invention relates to the pharmaceutical formulation for use in the treatment of pain and/or inflammation.

The term “pain” shall mean to include but is not limited to, nociceptive and neuropathic pain or combinations thereof; acute, intermittent and chronic pain; cancer pain; migraine and headaches of similar origin.

The term “inflammation” shall mean to include, but is not limited to, rheumatoid arthritis; osteoarthritis; and juvenile arthritis.

In the context of the present specification, the term “therapeutical” and “treatment” includes prevention and prophylaxis, unless there are specific indications to the contrary.

### BRIEF DESCRIPTION OF THE DRAWING

FIG. 1 shows an X-ray powder diffractogram for the crystalline form of 2-[2-(nitrooxy)ethoxy]ethyl {2-[(2,6-dichlorophenyl)amino]phenyl}acetate as obtained according to the process described in Example 5. (Form A of compound IVa)

The examples that follow will further illustrate the preparation of compounds of formula IV, expecially Form A of compound IVa, according processes described above. These examples are not intended to limit the scope of the invention as defined hereinabove or as claimed below.

### EXAMPLES

### Example 1

### Synthesis of 2-[2-(nitrooxy)ethoxy]ethyl (2-[(2,6-dichlorophenyl)amino]phenyl}acetate (compound of formula IVa).

2-(2-hydroxyethoxy)ethyl {2-[(2,6-dichlorophenyl)amino]phenyl}acetate (compound of formula IIa).

Diclofenac sodium (20 g, 63 mmol) was dissolved in diehyleneglycol (67 g, 0.63 mol) at 60° C. Toluene (170 mL) and conc. sulfuric acid (4.5 mL, 81.7 mmol) were added after the solids had dissolved. The reaction mixture was heated at 60° C. for 14 h before addition of K₂CO₃ (1 M, 120 mL). After phase separation the aqueous phase was discarded and the organic phase was washed with water (100 mL). The organic phase was concentrated under vacuum to give 23 g of Ia as a brown oil (85% yield, 90%-area HPLC-purity) to be used in the next step. MS [M⁺]=384; ¹H-NMR (CDCl₃) δ 7.34 (app d, J=8 Hz, 2H), 7.24 (app d, J=8 Hz, 1H), 7.12 (app t, J=7 Hz, 1H), 6.92-7.05 (m, 2H), 6.88 (br s, 1H), 6.54 (app d, J=8 Hz, 1H), 4.32 (app t, J=4 Hz, 2H), 3.85 (s, 2H), 3.64-3.76 (m, 4H), 3.50-3.58 (m, 2H), 2.08 (br s, 1H); ¹³C-NMR (CDCl₃) δ 172. 8, 143.1, 138.2, 131.1, 129.9, 129.4, 128.5, 124.6, 124.5, 123.5, 122.4, 118.7, 72.8, 69.3, 64.7, 62.10, 53.9, 38.9.
2-(2-Hydroxyethoxy)ethyl {2-[(2,6-dichlorophenyl)amino]phenyl}acetate (compound of formula IIa).

A mixture of Diclofenac Ia (450 g, 1.52 mol) and diethyleneglycol (2.42 kg, 22.8 mol) was stirred at 30° C. Thionyl chloride (90.1 g, 0.757 mol) was added over 30 min. After stirring for 6.5 h at 30° C., toluene (2.20 L) and aqueous potassium carbonate (168.1 g dissolved in 1800 mL of water, 1.22 mol) were added during continued stirring. After 0.5 h of agitation at inner temperature 29-30° C. the aqueous layer was separated off. The organic phase was washed three times with water (1.8 L per wash) at an inner temperature of 54-56° C. to improve phase separation. The organic phase was concentrated down under vacuum to a volume of 1900 mL. Before use in the following sulfonylation step (see below), toluene (0.70 L) was added and the water content of the resulting solution was measured by Karl Fisher-titration to be 0.07% w/w. Purity by HPLC: 92%-area.
2-{2-[(methylsulfonyl)oxy]ethoxy}ethyl {2-[(2,6-dichlorophenyl)amino]phenyl}acetate (compound of formula IIIa).

The hydroxiester IIa (23 g, 0.16 mol) isolated in the previous step was dissolved in toluene (300 mL) and N-methyl morpholine (16.9 g, 157 mmol) at 30° C. Methanesulfonyl chloride (18.0 g, 157 mmol) dissolved in toluene (50 mL) was added drop wise to the reaction. The reaction was heated to 60° C. over 2 h after which the reaction mixture was washed with 0.1 M sulfuric acid (200 mL) and water (2×200 mL). The organic phase was concentrated under reduced pressure and the resulting oil was dissolved in toluene (200 mL) and concentrated again. The crude product was dissolved in toluene (150 mL) at 30° C. and isooctane (150 mL) was added over 1 h before cooling to 5° C. After stirring the resulting slurry over night the crystals were filtered off, washed with isooctane (100 mL) and then dried at 40° C. under vacuum. This gave 52.4 g (71%) of the title compound as white crystals (98.0%-area HPLC-purity). Mp=87° C.; MS [M⁺]=462; ¹H-NMR (CDCl₃) δ 7.34 (app d, J=8 Hz, 2H), 7.23 (app d, J=7 Hz, 1H), 7.13 (app t, J=7 Hz, 1H), 6.97 (app q, J=8 Hz, 2H), 6.85 (br s, 1H), 6.54 (app d, J=8 Hz, 1H), 4.26-4.36 (m, 4H), 3.84 (s, 2H), 3.68-3.78 (m, 4H), 2.99 (s, 3H); ¹³C-NMR (CDCl₃) δ 172.2, 142.7, 137.7, 130.9, 129.5, 128.9, 128.1, 124.2, 124.1, 122.1, 118.3, 100.0, 69.1, 69.0, 64.1, 38.5, 37.6.
2-{2-[(Methylsulfonyl)oxy]ethoxy}ethyl {2-[(2,6-dichlorophenyl)amino]phenyl}acetate (compound of formula IIIa).

The solution of hydroxiester IIa (2.6 L) prepared in the previous step was mixed with N-methyl morpholine (154 g, 1.52 mol) before dropwise addition of methanesulfonyl chloride (174 g, 1.52 mol) at 30° C. over 25 min with efficient stirring. The inner temperature increased to 41° C. during the addition period. The reaction was stirred at 30° C. for another 40 min before increasing the temperature to 60° C. After stirring for 3 h 40 min more N-methyl morpholine (7.7 g, 76 mmol) and methanesulfonyl chloride (8.7 g, 76 mmol) were added and agitation at 60° C. was then continued for 54 min. Aqueous sulfuric acid (0.10 M, 1.8 L) was added at 60° C. and the resulting twophase system was stirred for about 20 min before phase separation. The organic layer was washed twice at 60° C. with water (2×1.8 L) and then concentrated under reduced pressure down to 1.4 L remaining volume. Isooctane (1.35 L) was added over 30 min at 60° C. before cooling to 30° C. After stirring the resulting slurry over night at 30° C. the crystals were filtered off and washed with isooctane (0.20 L). The obtained crystals were recrystallised once as described above from toluene (1.35 L) and isooctane (1.35 L). After filtration and washing with isooctane (0.90 L) the crystals were dried at 40° C. under vacuum. This gave 610.2 g (86.3% over two steps) of the title compound as white crystals (>99%-area HPLC-purity).
2-[2-(Nitrooxy)ethoxy]ethyl {2-[(2,6-dichlorophenyl)amino]phenyl}acetate (compound of formula IVa).

The mesylate IIIa (461 g, 0.997 mol) and lithium nitrate (293 g, 4.25 mol) were dissolved in N-methyl pyrrolidinone (1800 mL) and the temperature was set to 75° C. After 3.5 h another portion of lithium nitrate (146 g, 2.11 mol) was added. The reaction was run over night (total 27 h) before the reaction was stopped by decreasing to 35° C. and addition of toluene (1800 mL) and water (1000 mL). The water phase was separated off and the organic phase was washed with water (1000 mL). The organic phase was evaporated to dryness giving 513 g of IVa which solidified upon standing. An analytical sample (10 g) was recrystallised from n-butylacetate (30 mL) and isooctane (60 mL). Mp =73° C.; MS [M⁺]=429; ¹H-NMR (CDCl₃) δ 7.34 (app d, J=8, 2H) 7.24 (app d, J=8 Hz, 1H), 7.12 (app t, J=8 Hz, 1H), 6.97 (app q, J=8 Hz, 2H), 6.86 (br s, 1H), 6.55 (d, J=8 Hz, 1H), 4.54 (t, J=4 Hz, 2H), 4.30 (t, J=5 Hz, 2H), 3.84 (s, 2H), 3.66-3.74 (m, 4H); ¹³C-NMR (CDCl₃) δ 171.7, 142.2, 137.2, 130.4, 129.0, 128.4, 127.5, 123.7, 123.6, 121.5, 117.7, 71.4, 68.7, 66.6, 63.6, 38.0.
2-[2-(Nitrooxy)ethoxy]ethyl {2-[(2,6-dichlorophenyl)amino]phenyl}acetate (compound of formula IVa).

The mesylate IIIa (471 g, 1.02 mol) was mixed with n-butyl acetate (1.9 L) at 60° C. Tetrabutylammonium nitrate (62.3 g, 0.204 mol) and sodium nitrate (355 g, 5.15 mol), both ground using a mortar, were added at 60° C. and the resulting slurry was agitated at a jacket temperature of 60° C. for 10 min. Water (45.9 mL) was added and the jacket temperature was raised to 85° C. After 16 h 30 min of vigorous stirring the jacket temperature was raised to 90° C. and after a total of 51 h the mixture was cooled to 50° C. Water (1.9 L) was added and the resulting twophase system was stirred at 50° C. for 5 min. The water phase was separated off and the organic phase was washed twice with water (2×1.9 L) at 50° C. The organic phase was then evaporated down to a volume of 1.0 L. Isopropanol (2.36 L) was added at 50° C. and the resulting solution was cooled to an inner temperature of −11° C. over 15 h. The formed crystals were filtered off and washed with isopropanol (1.0 L) and then dried under vacuum at 40° C., to give 361.6 g (82.7%) of pure IVa. The purity according to HPLC was 98 area-%.
2-[2-(Nitrooxy)ethoxy]ethyl {2-[(2,6-dichlorophenyl)amino]phenyl}acetate (compound of formula IVa).

The mesylate IIIa (608.8 g, 1.317 mol) and tetrabutylammonium nitrate (120.8 g, 0.397 mol) were mixed with n-butyl acetate (1.7 L) at 60° C. Acetonitrile (0.70 L) and sodium nitrate (459.7 g, 6.668 mol) were added at 60° C. and the resulting slurry was agitated at a jacket temperature of 87° C. for 50 h. Water (2.4 L) was added and the jacket temperature was lowered to 50° C. After 10 min of stirring the water phase was separated off and the organic phase was washed twice with water (2×2.4 L) at 50° C. The organic phase was then evaporated down to a volume of 1.5 L. Isopropanol (3.1 L) was added at 50° C. and the resulting solution was cooled to an inner temperature of −12° C. over 15 h. After 7 h of stirring at −12° C. the formed crystals were filtered off and washed with isopropanol (0.84 L) and then dried under vacuum at 40° C., to give 527.7 g (93.4%) of pure IVa. The purity according to HPLC was >99 area-%.

### Example 2

### Synthesis of 4-(nitrooxy)butyl {2-[(2,6-dichlorophenyl)amino]phenyl}acetate (compound of formula IVb)

4-Hydroxybutyl {2-r(2,6-dichlorophenyl)amino]phenyl}acetate (compound of formula IIb).

To a mixture of Diclofenac sodium (20.0 g, 62.9 mmol) and 1,4-butanediol (56.6 g, 629 mmol) in toluene (120 mL) at 65° C. was added sulfuric acid (4.5 mL, 84.5 mmol). The resulting clear solution was stirred at 65° C. over 6 h before cooling to 50° C. The reaction mixture was washed with aqueous potassium bicarbonate (0.2 M, 120 mL) and water (2×120 mL). After phase separation the toluene was evaporated giving 22.9 g IIb as a brown oil (88%, HPLC purity of at least 89%-area), which was used in the next step. ¹H-NMR (CDCl₃) δ 7.34 (app d J=8 Hz, 2H), 7.23 (app d, J=8 Hz, 1H), 7.13 (app t, J=7 Hz, 1H), 6.97 (app q, J=8 Hz, 2H), 6.56 (app d, J=8 Hz, 1H), 4.19 (t, J=7 Hz, 2H), 3.82 (s, 2H), 3.63 (t, J=7Hz, 2H), 1.71-1.80 (m, 2 H), 1.55-1.64 (m, 2H); ¹³C-NMR (CDCl₃) δ 172.4, 142.6, 137.7, 130.8, 129.4, 128.8, 127.9, 124.4, 124.0, 121.9, 118.2, 65.1, 62.1, 38.6, 28.9, 25.0.
4-[(Methylsulfonyl)oxy]butyl {2-[(2,6-dichlorophenyl)amino]phenyl}acetate (compound of formula IIIb).

The ester IIb (20 g, 54 mmol) from the previous step and methanesulfonyl chloride (7.5 g, 65.1 mmol) were dissolved in toluene (100 mL) at 20° C. N-Methylmorpholine (6.0 g, 59.7 mmol) was added drop wise. After complete addition the solution (slightly cloudy) was heated at 40° C. over 5 h. Toluene was added (40 mL) and the reaction was heated at 60° C. for 0.5 h before addition of sulfuric acid (aq) (0.1 M, 80 mL). The aqueous layer was discarded and the toluene phase was washed with aqueous potassium carbonate (0.6 M, 40 mL) before evaporation of the toluene to give 35 g of an oil. The resulting oil was dissolved in toluene (60 mL) at room temperature and isooctane was added. The obtained slurry was cooled down to 5° C., the crystals were filtered off and washed with isooctane. The crystals were allowed to dry under suction for 1 h. This gave 19.0 g of IIIb as white crystals (79% yield with a HPLC purity of 98.9%-area). Mp=57-58° C. ¹H-NMR (CDCl₃) δ 7.35 (app d, J=8 Hz, 2H), 7.22 (app d, J=8 Hz, 1H), 7.13 (app t, J=7 Hz, 1H), 6.93-7.01 (m, 2H), 6.88 (br s, 1H), 6.55 (app d, J=8 Hz, 1 H), 4.15-4.28 (m, 4H), 3.81 (s, 2H), 2.99 (s, 3H), 1.74-1.84 (m, 4H); ¹³C-NMR (CDCl₃) δ 172.3, 142.7, 137.7, 130.8, 129.5, 128.9, 128.0, 124.2, 124.1, 122.0, 118.3, 69.1, 64.3, 38.6, 64.3, 38.6, 37.4, 25.8, 24.8.
4-(Nitrooxy)butyl {2-[(2,6-dichlorophenyl)amino]phenyl}acetate (compound of formula IVb).

Compound IIIb (5.0 g, 11 mmol) and lithium nitrate (2.2 g, 32 mmol) were dissolved in N-methylpyrrolidinone (15 mL) at 70° C. After 23 h the reaction was cooled to 35° C., toluene (20 mL) was added and the reaction was washed with water (2×30 mL). The organic layer was dried over Na₂SO₄ and evaporated to dryness. The resulting oil was purified by silica gel chromatography (EtOAc: Hexane; 80:20) and 4.02 g of IVb as a colorless oil was collected. ¹H-NMR (CDCl₃) δ 7.34 (app d, J=8 Hz, 2H), 7.22 (app d, J=7 Hz, 1H), 7.08-7.19 (m, 1H), 6.91-7.02 (m, 2H), 6.88 (br s, 1H), 6.55 (app d, J=7 Hz, 1H), 4.38-4,46 (m, 2H), 4.14-4.21 (m, 2H), 3.81 (s, 2H), 1.71-1.82 (m, 4H); ¹³C-NMR (CDCl₃) δ 172.3, 142.7, 137.8, 130.8, 129.5, 128.9, 128.1, 124.2, 124.1, 122.1, 118.3, 72.5, 64.3, 38.6, 25.0, 23.5.

### Example 3

### Synthesis of 2-{2-[2-(nitrooxy)ethoxy]ethoxy}ethyl {2-[(2,6-dichlorophenyl-)amino]-phenyl}acetate (compound of formula IVc).

2-[2-(2-Hydroxyethoxy)ethoxy]ethyl {2-[(2,6-dichlorophenyl)amino]phenyl}acetate (compound of formula IIc).

Thionyl chloride (1.2 mL, 16.9 mmol) was added to a suspension of Diclofenac (10 g, 33.8 mmol) and triethylene glycol (90 mL, 676 mmol) at 30° C. The reaction was stirred for 7 h before addition of aqueous potassium carbonate (0.27 M, 100 mL) and toluene (100 mL). The temperature was increased to 60° C. and the water phase was discarded. The organic phase was washed with water (3×100 mL) and concentrated to give 14.4 g of IIc as an oil. This oil was used directly in the next step. ¹H-NMR (CDCl₃) δ 7.33 (app d, J=8 Hz, 2H) 7.23 (app d, J=7 Hz, 1H), 7.08-7.20 (m, 1H), 6.85-7.07 (m, 3H), 6.54 (app d, J=8 Hz, 1H), 4.31 (app t, J=5 Hz, 2H), 3.85 (s, 2H), 3.71 (m, 4 Hz, 4H), 3.54-3.64 (m, 4H), 2.50 (app br s, 1H); ¹³C-NMR (CDCl₃) δ 172.4, 142.8, 137.8, 130.9, 129.6, 128.9, 128.01, 124.2, 124.1, 122.0, 118.2, 72.5, 70.6, 70.3, 69.0, 64.3, 61.7, 38.5.
10, 10-Dioxido-3,6,9-trioxa-10-thiaundec-1-yl {2-[(2,6-dichlorophenyl)amino]-phenyl}acetate (compound of formula IIIc).

The hydroxiester IIc (13.4 g, 31.3 mmol) from the previous step was dissolved in toluene (80 mL) together with N-methylmorpholine (3.5 g, 34.4 mmol) at 30° C. Methanesulfonyl chloride (3.9 g, 34.4 mmol) in toluene (10 mL) was added over 15 min. After complete addition the temperature was increased to 60° C. for 2 h and then lowered again to 30° C. overnight. Aqueous sulfuric acid (0.1 M, 40 mL) was added and the temperature was increased to 60° C. for the extraction. The water phase was discarded and the organic phase was washed with water (2×100 mL). The organic phase was concentrated to give an oil (15.3 g). This oil was purified by chromatography on silica (EtOAc/hexane; 30/70 to 50/50) to give 13.8 g of IIIc as a brown oil. ¹H-NMR (CDCl₃) δ 7.34 (app d, J=8 Hz, 2H) 7.23 (app d, J=7 Hz, 1H), 7.12 (app t, J=7 Hz, 1H) 6.88-7.02 (m, 2H), 6.54 (d, J=8 Hz, 1H), 4.75-4.36 (m, 4H), 3.84 (s, 2H), 3.67-3.74 (m, 4H) 3.6 (app br s, 4 H), 3.04 (s, 3H); ¹³C-NMR (CDCl₃) δ 172.2, 142.6, 137.6, 130.8, 129.4, 128.8, 127.9, 124.1, 124.0, 121.9, 118.1, 70.4, 69.1, 68.91, 68.87, 64.2, 60.2, 38.4, 37.5.
2-{2-[2-(Nitrooxy)ethoxy]ethoxy}ethyl {2-[(2,6-dichlorophenyl)amino]phenyl}acetate (compound of formula IVc).

Sodium nitrate was added to a solution of the mesylate IIIc from the previous step (12.7 g, 25.1 mmol) and tetrabutylammonium nitrate (2.3 g, 7.6 mmol) in n-butylacetate (50 mL) and water (1.7 mL) at 60° C. The resulting suspension was heated to 85° C. for 41 h before cooling to 60° C. and addition of water (100 mL). After extraction the water phase was separated off and the organic phase was washed twice with water (2×100 mL). The organic phase was evaporated to dryness and the residue was crystallised from n-butylacetate (26 mL) and 2-propanol (110 mL). The crystals were filtered off, washed with 2-propanol (25 mL) and dried under reduced pressure at 40° C. to give 9.3 g of IVc as crystals. Mp=68° C. ¹H-NMR (CDCl₃) δ 7.34 (app d, J=8 Hz, 2H) 7.23 (app d, J=7 Hz, 1H), 7.12 (app t, J=7 Hz, 1H), 6.91-7.02 (m, 3H), 6.55 (app d, J=8 Hz, 1H), 4.58 (app t, J=5 Hz, 2H), 4.31 (app t, J=4 Hz, 2H), 3.85 (s, 2H), 3.67-3.78 (m, 4H), 3.60 (app s, 4H); ¹³C-NMR (CDCl₃) δ 172.4, 142.8, 137.8, 130.9, 129.5, 128.9, 128.0, 124.3, 124.0, 122.0, 118.3, 72.2, 70.8, 70.6, 69.1, 67.2, 64.3, 38.5

### Example 4

### Synthesis of 3-(nitrooxy)propyl 2-(2-benzoylphenyl)propanoate (compound of formula IVd).

3-Hydroxypropyl (2S)-2-(2-benzoylphenyl)propanoate (compound of formula IId)

A mixture of (S)-ketoprofen (10.0 g, 39.3 mmol), 1,3-propanediol (29.9 g, 393 mmol), toluene (40 mL) and conc. sulfuric acid (0.3 g, 3.06 mmol) were heated to 80-95° C. for 28 h before cooling to 45° C. and addition of a 5% aqueous potassium carbonate solution (50 mL). The bottom aqueous layer was separated off and the top organic layer was washed with water (2×50 mL). The organic layer was concentrated down to dryness under reduced pressure to give 11.9 g of IId as a colorless oil (96%-area LC-purity). The enantiomeric purity was >99.5%-area. MS [M⁺]=312, ¹H-NMR (CDCl₃) δ 7.78 (app t, J=7Hz, 3H), 7.41-7.68 (m, 6H), 4.30-4.79 (m, 2H), 3.81 (q, J=7 Hz, 1H), 3.51 (t, J=6 Hz, 2H), 2.35 (br s, 1H), 1.82 (quin, J=7 Hz, 2H), 1.53 (d, J=7 Hz, 3H); ¹³C-NMR (CDCl₃) δ 196.7, 174.4, 140.9, 137.9, 137.4, 132.6, 131.5, 130.1, 129.1, 128.6, 128.3, 61.9, 58.9, 45.4, 31.5, 18.4, 14.2.
3-[(methylsulfonyl)oxy]propyl (2S)-2-(2-benzoylphenyl)propanoate (compound of formula IIId).

The hydroxiester IId (5.0 g, 16 mmol) from the previous step was dissolved in toluene (25 mL). Methanesulfonyl chloride (2.2 g, 19.2 mmol) was added to the mixture followed by dropwise addition of N-methylmorpholine (1.78 g, 17.6 mmol). The reaction mixture was heated at 40° C. for 1 h and then heated to 60° C. before addition of aqueous sulfuric acid (0.1 M, 20 mL) and toluene (10 mL). After extraction the mixture was separated and the organic layer was washed with aqueous potassium carbonate (0.93 g in 20 mL of water). The organic layer was concentrated under vacuum to give 5.6 g of IIId as an oil. MS [M⁺]=391; ¹H-NMR (300 MHz, CDCl₃) δ 7.78 (app t, J=7 Hz, 3H), 7.41-7.69 (m, 6H), 4.21 (app t, J=6 Hz, 2H), 4.18 (app t, J=6 Hz, 2H), 3.82 (q, J=7 Hz, 1H), 2.94 (s, 3H), 2.04 (quin, J=7 Hz, 2H), 1.55 (d, J=7 Hz, 3H); ¹³C-NMR (100 MHz, CDCl₃) δ 196.4, 173.8 140.7, 138.0, 132.5, 131.4, 130.0, 129.1, 129.0, 128.6, 128.3, 66.0, 60.4, 45.3, 37.2, 28.4, 18.2.
3-(nitrooxy)propyl (2S)-2-(2-benzoylphenyl)propanoate (compound of formula IVd).

A mixture of the mesylate IIId (5.0 g, 12.8 mmol) from the previous step and lithium nitrate (2.65 g, 38.5 mmol) in N-methyl pyrrolidinone (15 mL) was heated at 70° C. for 9 h. The heating was removed and the reaction mixture was allowed to reach room temperature before addition of toluene (30 mL) and water (20 mL). The layers were separated and the organic layer was washed with water (20 mL). Concentration to dryness gave IVd as an oil (5.0 g). The enantiomeric purity was 99.5%-area. MS [M⁺]=357; ¹H-NMR (300 MHz, CDCl₃) δ 7.73-7.84 (m, 3H), 7.67 (app d, J=7 Hz, 1H), 7.38-7.64 (m, 5H), 4.40 (t, J=6 Hz, 2H), 4.18 (t, J=6 Hz, 2H), 3.81 (q, J=7 Hz, 1H), 2.94 (s, 3H), 2.01 (quin, J=6 Hz, 2H), 1.55 (d, J=7 Hz, 3H); ¹³C-NMR (100 MHz, CDCl₃) δ 196.4, 173.8, 140.7, 138.0, 137.5, 132.6, 131.4, 130.0, 129.2, 129.1, 128.6, 128.3, 69.6, 60.8, 45.3, 26.3, 18.3.

### Example 5

X-ray powder diffraction analysis (XRPD) was performed according to standard methods, for example those described in Giacovazzo, C. et al (1995), pp 287-301, Fundamentals of Crystallography, Oxford University Press; Jenkins, R. and Snyder, R. L. (1996), Introduction to X-Ray Powder Diffractometry, John Wiley & Sons, New York; Bunn, C. W. (1948), pp 103-127, Chemical Crystallography, Clarendon Press, London; or Klug, H. P. & Alexander, L. E. (1974), X-ray Diffraction Procedures, second edition, John Wiley and Sons, New York.

X-ray analyses were performed using a Philips X'Pert MPD diffractometer.

Differential scanning calorimetry (DSC) was performed using a Perkin Elmer DSC7 instrument, according to standard methods, for example those described in Höhne, G. W. H. et al (1996), Differential Scanning Calorimetry, Springer, Berlin.

Thermogravimetric analysis (TGA) was performed using a Perkin Elmer TGA7 instrument.

The crystal form prepared in accordance with Example 1 below showed essentially the same XRPD diffraction pattern and DSC and TGA thermograms as the crystal forms prepared according to the other Examples disclosed belowthereby allowing for experimental error. The limits of experimental error for DSC onset temperatures may be in the range ±5° C. (e.g. ±2° C.), and for XRPD distance values may be in the range ±2 on the last decimal place.

### Synthesis of the anhydrate of 2-[2-(nitrooxy)ethoxy]ethyl f2-[(2,6-dichlorophenyl)amino]phenyl}acetate

### Example 5a

0.3 g of 2-[2-(nitrooxy)ethoxy]ethyl {2-[(2,6-dichlorophenyl)amino]phenyl}acetate IVa was charged together with 0.9 ml toluene into a 4 ml test tube. The test tube was placed on a magnetic stirrer at ambient temperature. After all compound was dissolved, 1.8 ml isooctane was added 0.3 ml-wise. Crystallization started after all isooctane had been added. 4.5 h after crystallization had started the crystals were filtered under vacuo. The tube was rinsed with 0.3 ml isooctane. The crystals were thereafter dried in a vacuum oven at 35° C. The yield (based on the amount left in the mother liquor) was 80.6%.

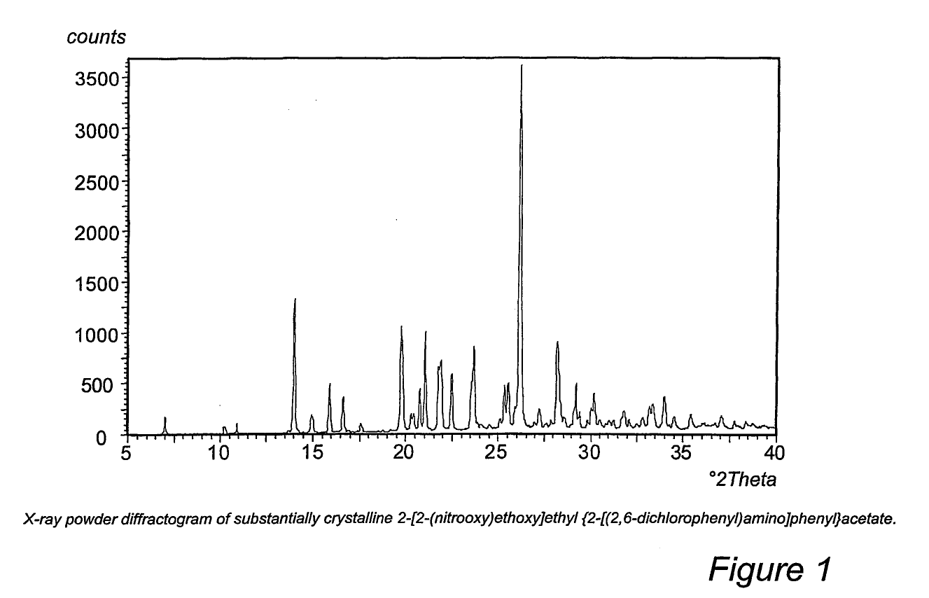

The crystals were analyzed by XRPD, DSC and TGA. The XRPD gave the result tabulated in Table 1 and shown in FIG. 1. The DSC thermogram showed a sharp melting point at 72° C. and the TGA thermogram showed that the crystal did not contain any significant amounts of solvents impurities.

TABLE 1
X-ray powder diffraction data for
2-[2-(nitrooxy)ethoxy]ethyl{2-[(2,6-
dichlorophenyl)amino]phenyl}acetate.

| D/Å | Relative |
|---|---|
| 12.7 | M |
| 8.7 | W |
| 8.1 | W |
| 6.3 | S |
| 5.94 | M |
| 5.91 | M |
| 5.58 | M |
| 5.34 | M |
| 5.05 | W |
| 4.50 | S |
| 4.48 | S |
| 4.38 | M |
| 4.35 | M |
| 4.28 | M |
| 4.23 | S |
| 4.08 | S |
| 4.06 | S |
| 3.96 | S |
| 3.78 | S |
| 3.76 | S |
| 3.55 | W |
| 3.52 | M |
| 3.49 | M |
| 3.44 | W |
| 3.41 | VS |
| 3.31 | W |
| 3.28 | M |
| 3.17 | S |
| 3.15 | S |
| 3.13 | W |
| 3.06 | M |
| 3.04 | W |
| 2.97 | M |
| 2.96 | M |
| 2.81 | W |
| 2.70 | M |
| 2.68 | M |
| 2.64 | M |
| 2.60 | W |
| 2.54 | W |
| 2.43 | W |

The main peaks, with positions (D/Å) and relative intensities have been extracted from the diffractogram in FIG. 1. The relative intensities are given as VS=Very Strong, S=Strong, M=medium, W=Weak. Only peaks below 2θ=40° have been included. Some additional very weak peaks found in the diffractogram have been omitted from the table but are presented in FIG. 1.

All peaks can be indexed with the monoclinic unit cell: a=13.79 Å, b=11.90 Å, c=13.01 Å, α=90°, β=94.0°, γ=90°.

### Example 5b

0.3 g of IVa was charged together with 0.9 ml methyl isobutyl ketone into a 4 ml test tube. The test tube was placed on a magnetic stirrer at ambient temperature. Additional 0.3 ml 4-methyl-2-pentanone was necessary to dissolve all compound. Thereafter 1.8 ml isooctane was added 0.3 ml-wise. Crystallization started after all isooctane had been added. 4 h after crystallization had started the crystals were filtered under vacuo. The tube was rinsed with 0.3 ml isooctane. The crystals were thereafter dried in a vacuum oven at 35° C. The yield (based on the amount left in the mother liquor) was 44.1%.

The crystals were analyzed by XRPD, DSC and TGA. The results were essentially the same as those exhibited by the form obtained according to Example 5a.

### Example 5c

2.5 g of IVa was charged together with 7.5 ml butyl acetate into a 100 ml jacketed reactor. The reactor was heated to 35° C. to dissolve all compound. Thereafter a temperature profile was started: the temperature was lowered to 20° C. in 1.5 h and then kept for 0.5 h at 20° C. At 20° C. 15 ml isooctane was added dropwise. Crystallization started after 12 ml isooctane was added. The temperature was lowered further to 0° C. in 3 h. After 0.5 h at 0° C. the crystals were filtered under vacuo. The reactor was rinsed with 7.5 ml cooled isooctane. The crystals were thereafter dried in a vacuum oven at 35° C. The yield (based on the amount left in the mother liquor) was 91.6%.

The crystals were analyzed by XRPD, DSC, TGA, LC, and GC. The results from XRPD, DSC and TGA were essentially the same as those exhibited by the form obtained according to Example 5a. LC showed a purity of 99.12 area %, GC showed 0.01 w/w % isooctane and 0.10 w/w % butylacetate. The starting material had a purity of 98.42 area % and contained 0.13 w/w % ethyl acetate.

### Example 5d

0.5 g of IVa was charged together with 1.5 ml tert-butyl methyl ether into a 4 ml test tube. The tube was placed into an oil-bath. Agitation was provided by a magnetic stirrer. The oil bath was heated until a clear solution was obtained in the test tube. This was the case at 40° C. Thereafter the oil bath temperature was again lowered to 20° C. The mixture was held stirred over night and crystals were formed. The crystals were filtered under vacuo. The tube was rinsed with 0.3 ml tert-butyl methyl ether. The crystals were thereafter dried in a vacuum oven at 35° C. The yield (based on the amount left in the mother liquor) was 77%.

The crystals were analyzed by XRPD, DSC and TGA. The results were essentially the same as those exhibited by the form obtained according to Example 1. The results showed essentially the same XRPD pattern as those exhibited by the form obtained according to Example 5a.

### Example 5e

0.5 g of IVa was charged together with 1.5 ml butanol into a 4 ml test tube. The tube was placed in an oil-bath. Agitation was provided by a magnetic stirrer. The oil bath was heated until a clear solution was obtained in the test tube. This was the case at 60° C. Thereafter the test tube was placed on a magnetic stirrer at ambient temperature. Crystallization started immediately. After 2.5 h the crystals were filtered under vacuo. The tube was rinsed with 0.3 ml butanol. The crystals were thereafter dried in a vacuum oven at 35° C. The yield (based on the amount left in the mother liquor) was 94%.

The crystals were analyzed by XRPD, DSC and TGA. The results were essentially the same as those exhibited by the form obtained according to Example 5a.

### Example 5f

0.5 g of IVa was charged together with 1.5 ml isopropanol into a 4 ml test tube. The tube was placed in an oil-bath. Agitation was provided by a magnetic stirrer. The oil bath was heated until a clear solution was obtained in the test tube. This was the case at 60° C. Thereafter the test tube was placed on a magnetic stirrer at ambient temperature. Crystallization started immediately. After 2.5 h the crystals were filtered under vacuo. The tube was rinsed with 0.3 ml isopropanol. The crystals were thereafter dried in a vacuum oven at 35° C. The yield (based on the amount left in the mother liquor) was 96%.

The crystals were analyzed by XRPD, DSC and TGA. The results were essentially the same as those exhibited by the form obtained according to Example 5a.

### Example 5g

0.5 g of IVa was charged together with 2.5 ml ethanol into a 4 ml test tube. The test tube was placed on a magnetic stirrer at ambient temperature. The slurry in the test tube was stirred over night. The crystals were filtered under vacuo. The tube was rinsed with 0.6 ml ethanol. The crystals were thereafter dried in a vacuum oven at 35° C. The yield (based on the amount left in the mother liquor) was 93.4%.

The crystals were analyzed by XRPD, DSC and TGA. The results were essentially the same as those exhibited by the form obtained according to Example 5a.

### Example 5h

0.5 g of IVa was charged together with 2.5 ml isooctane into a 4 ml test tube. The test tube was placed on a magnetic stirrer at ambient temperature. The slurry in the test tube was stirred over night. The crystals were filtered under vacuo. The tube was rinsed with 0.3 ml isooctane. The crystals were thereafter dried in a vacuum oven at 35° C. The yield (based on the amount left in the mother liquor) was 99.1%.

The crystals were analyzed by XRPD, DSC and TGA. The results were essentially the same as those exhibited by the form obtained according to Example 5a.

### Example 5i

Compound IVa (4.0 g) was mixed with acetone (8.0 mL) and the resulting mixture was stirred at 40° C. When a clear solution was obtained, isopropanol (40 mL) was added and the solution was left stirring over night at ambient temperature. The solution was then seeded at ambient temperature and after about 30 min the seed was still undissolved. The temperature was then lowered from 20° C. to −5° C. over 12 hours. The crystals were filtered off and dried under vacuum at 40° C. to give 3.55 g (88.8%) of pure IVa. The crystals were analyzed by XRPD and HPLC and the results show essentially the same XRPD pattern as those exhibited by the form obtained according to Example 5a. HPLC showed a purity of 98.2 area %.

### Example 5j

Compound IVa (10.0 g) was mixed with acetonitrile (62 mL) and the resulting mixture was stirred at room temperature. When a clear solution was obtained, water (14 mL) was added and the obtained solution was then seeded at ambient temperature. Water (2 mL) was added and after about 1 h 30 min of stirring the seed was still undissolved. The solution was left stirring for two days at ambient temperature and after that the temperature was lowered to −10° C. over 24 hours. The crystals were filtered off, washed with water (20 mL) and dried under vacuum at 40° C. to give 7.98 g (79.8%) of pure IVa. The crystals were analyzed by XRPD and HPLC and the results show essentially the same XRPD pattern as those exhibited by the form obtained according to Example 5a. HPLC showed a purity of 99.0 area %.

### Example 5k

Compound IVa (10.3 g) was mixed with ethyl acetate (20 mL) and the resulting mixture was stirred at 40° C. When a clear solution was obtained, isopropanol (80 mL) was added and the temperature was lowered from 40° C. to −10° C. over 15 hours. The crystals were filtered off, washed with isopropanol (20 mL) and dried under vacuum at 40° C. to give 9.37 g (91%) of pure IVa. The crystals were analyzed by XRPD and HPLC and the results show essentially the same XRPD pattern as those exhibited by the form obtained according to Example 5a. HPLC showed a purity of 99 area %.

### Example 5I

Compound IVa (438.9 g) was mixed with acetone (4.0 L) and the resulting mixture was stirred at 30° C. until a clear solution was obtained. When a clear solution was obtained, water (1.3 L) was added and the temperature was lowered from 30° C. to −3° C. over 8 hours. After stirring at −3° C. for 10 h the temperature was further lowered to −12° C. over 5 h. The crystals were then filtered off, washed with water (0.90 L) and dried under vacuum at 40° C. to give 392 g (89.2%) of pure IVa. The crystals were analyzed by XRPD and HPLC and the results show essentially the same XRPD pattern as those exhibited by the form obtained according to Example 5a. HPLC showed a purity of >99 area %.

Abbreviations:

D distance measured in Å [Ångström]
DSC differential scanning calorimetry
FT-IR Fourier-transformed infrared spectroscopy
NMR Nuclear magnetic resonance
TGA thermogravimetric analysis
XRDP X-ray powder diffractogram

1. A process for the manufacturing manufacture of NO-donating compounds comprising; (1), 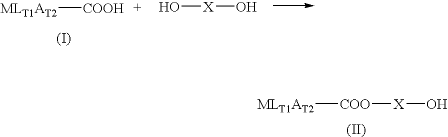
using an acidic or dehydrating agent and a first solvent, optionally followed by purification using extraction or crystallization, and 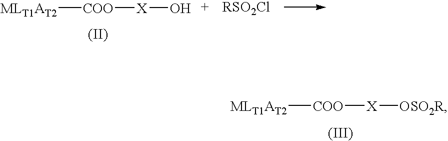
using a second solvent, a base and optionally a catalyst, followed by purification using extraction and crystallization, and (3), 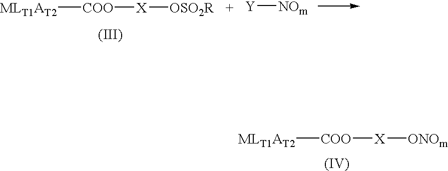
using a third solvent and optionally a catalyst,
optionally followed by a crystallization process for obtaining the compound of formula IV in a substantially crystalline form, and wherein:
M is a radical of a physiologically active compound;
L is O, S, (CO)O, (CO)NH, (CO)NR¹, NH, NR¹, wherein R¹ is a linear or branched alkyl group, or 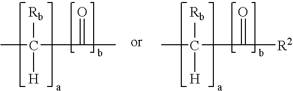
wherein R_{b} is H, C₁₋₁₂alkyl or C₂₋₁₂alkenyl;
R² is (CO)NH, (CO)NR¹, (CO)O, or CR¹ and a and b are independently 0 or 1;
A is a substituted or unsubstituted straight or branched alkyl chain;
X is a carbon linker;
R is selected from the group consisting of C₁-C₈ alkyl, phenyl, phenylmethyl,
C₁-C₄ alkylphenyl, halophenyl, nitrophenyl, acetylaminophenyl, halogen, CF₃ and n-C₄F₉;
Y—NO₃ is selected from the group consisting of lithium nitrate, sodium nitrate, potassium nitrate, magnesium nitrate, calcium nitrate, iron nitrate, zinc nitrate and tetraalkylammonium nitrate, wherein alkyl is a straight or branched C₁-C₁₈-alkyl;
m is 1 or 2; and
T1and T2 are each independently 0, 1, 2 or 3;
with the proviso that
when ML_{T1}A_{T2}-COOH is naproxen, then X is not (CH₂)₄.

2. The process according to claim 1, wherein group M is part of a molecule of an NSAID, COX 1 or COX 2 inhibitor.

3. The process according to claim 1, wherein X is selected from the group consisting of
linear —(CH₂)_{w1}—, wherein w1 is an integer of from 2 to 6; —(CH₂)₂—O—(CH₂)₂—, and —CH₂—C₆H₄—CH₂—.

4. The process according to claim 1, wherein R is selected from the group consisting of
C₁-C₈-alkyl, phenyl, phenylmethyl, C₁-C₄-alkylphenyl, halophenyl, nitrophenyl, acetylaminophenyl, and halogen.

5. The process according to claim 1, wherein the group ML_{T1}A_{T2} is selected from the group consisting of:

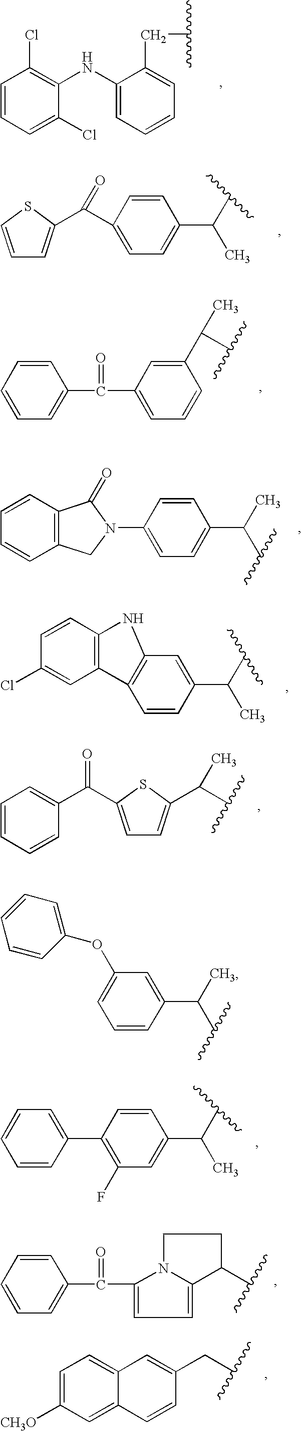

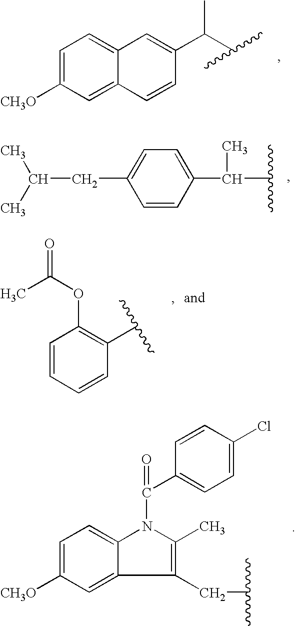

6. The process according to claim 5, wherein the group ML_{T1}A_{T2} is:

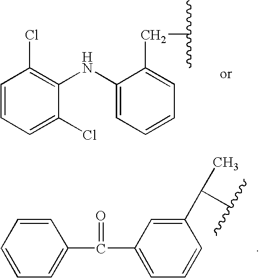

7. The process according to claim 1, wherein the crystallization process for the compound of formula IV comprises:
a) i) dissolving the compound in a fourth solvent; or,
ii) extracting the compound from the reaction solution into a fourth solvent; or,
iii) starting from the reaction solution comprising the compound;
b) evaporating the fourth solvent;
c) adding an anti-solvent and/or cooling
d) isolating the crystals formed, and optionally;
e) recrystallizing the crystals formed or isolated.

8. The process according to claim 7, wherein the crystallization process for compound 2-[2-(nitrooxy)-ethoxy]ethyl{2-[(2,6-dichlorophenyl)amino]phenyl}acetate (IVa) comprises:
a) extracting the compound from the reaction solution into a the fourth solvent;
b) evaporating the fourth solvent;
c) adding an anti-solvent and/or cooling;
d) isolating the crystals formed, and optionally;
e) recrystallizing the crystals formed or isolated.

9. The process according to claim 1, wherein the acidic or dehydrating agent is selected from the group consisting of sulphuric acid or its salts, perchloric acid polystyrene sulphonic acids, zeolites, acidic clays, sand in combination with strong hydrophilic acids, and montmorillonites.

10. The process according to claim 1, wherein the first solvent is a non-polar and/or non acidic solvent.

11. The process according to claim 1, wherein the second solvent is selected from a group consisting of toluene, cumene, xylenes, ethyl acetate, acetonitrile, butyl acetate, and isopropyl acetate.

12. The process according to claim 1, wherein the base is triethylamine or N-methylmorpholine.

13. The process according to claim 1, wherein the catalyst is 4-(dimethylamino)pyridine.

14. The process according claim 1, wherein the compound of formula III is crystallized from an organic solvent.

15. The process according to claim 14, wherein an anti-solvent is used in the crystallization of compound of formula III.

16. The process according to claim 1, wherein Y—NO₃ is selected from the group consisting of lithium nitrate, sodium nitrate, potassium nitrate, magnesium nitrate, calcium nitrate, and mixtures thereof.

17. The process according to claim 1, wherein the third organic solvent is selected from the group consisting of N-methylpyrrolidinone, sulpholane, tetramethylurea, 1,3-dimethyl-2-imidazolidinone, acetonitrile, methyl isobutylketone, ethyl acetate, butyl acetate, isopropyl acetate, and mixtures thereof.

18. The process according to claim 1, wherein the phase transfer-catalyst is selected from the group consisting of tetraalkylammonium salt, arylalkylammonium salt, tetraalkylphosphonium salt, arylalkylphosphonium salt, crown ether, pentaethylene glycol, hexaethylene glycol, polyethylene glycols, and mixtures thereof.

19. The process according to claim 7, wherein the fourth solvent is selected from the group consisting of lower alkyl acetates, lower alkyl alcohols, aliphatic hydrocarbons, aromatic hydrocarbons, heteroaromatic hydrocarbons, dialkyl ketones, dialkyl ethers, nitriles, water, and mixtures thereof.

20. The process according to claim 7, wherein the anti-solvent is selected from the group consisting of ethanol, 2-propanol, toluene, cumene, xylenes, ligroin, petroleum ether, halobenzenes, heptanes, hexanes, octanes, cyclohexanes, cycloheptanes, and mixtures thereof.

21. The process according to claim 7, wherein the solvent in step d) is selected from the group consisting of toluene, cumene, xylenes, methyl iso-butyl ketone, di-n-butyl ether, tert-butyl methyl ether, tetrahydrofuran, acetonitrile, n-butyl acetate, dichloromethane, and mixtures thereof.

22. The process according to claim 1, wherein the process is conducted at a temperature between −40° C. and 120° C.

23. A process for the manufacturing manufacture of NO donating diclofenac of formula IVa, IVb or IVc, comprising:
(1), reacting a compound of formula Ia with HO—X—OH, wherein X is C₂H₄OC₂H₄, C₄H₈, or C₂H₄OC₂H₄OC₂H₄, to obtain compounds of formula IIa, IIb or IIc, 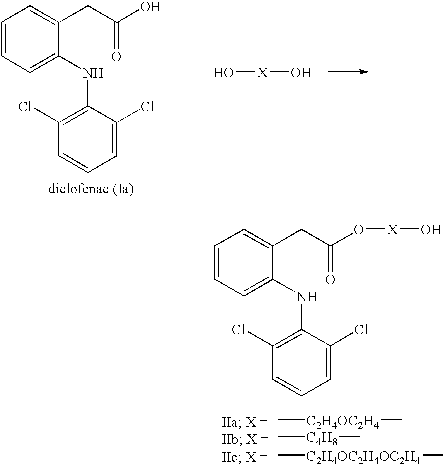
(2), reacting the compounds of formula IIa, IIb or IIc with RSO₂Cl, wherein R is defined in claim 1, to obtain compounds of formula IIIa, IIIb or IIIc, 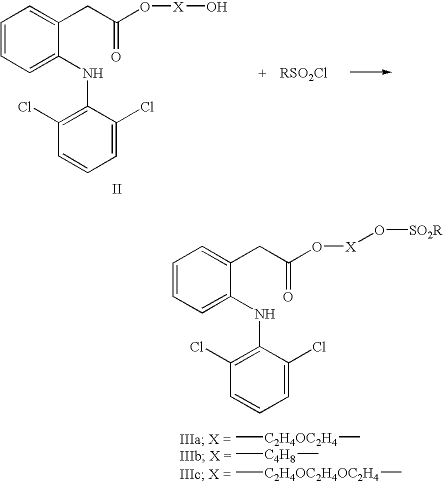
(3), reacting the compounds of formula IIIa, IIIb or IIIc with a nitrate source Y—NO₃, wherein Y is defined in claim 1, to obtain compounds of formula IVa, IVb or IVc, 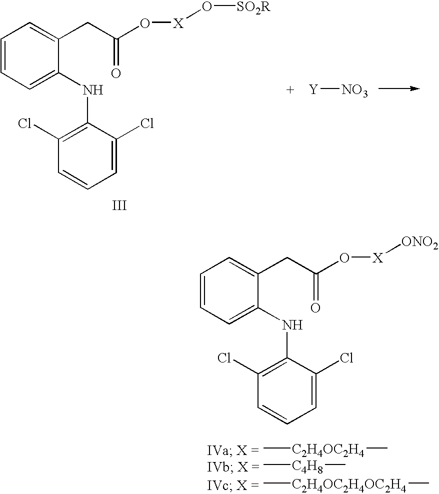
crystallizing the compounds of formula IVa, IVb or IVc by:
a) extracting the compound from the reaction solution into a solvent;
b) evaporating the solvent;
c) adding an anti-solvent and/or cooling
d) isolating the crystals formed, and optionally;
e) recrystallizing the crystals formed or isolated.

24. The process according to claim 1, wherein the chemical purity of Form A of compound IVa is above 95%.

25. A process for the manufacture of NO donating ketoprofen of formula IVd comprising:
(1), reacting a compound of formula Id with 1,3-propanediol to obtain a compound of formula IId, 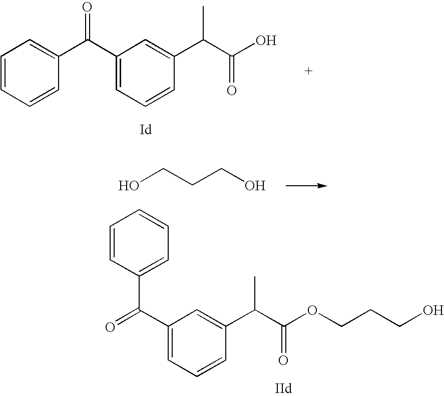
(2), reacting the compound of formula lid with RSO₂Cl, wherein R is as defined in claim 1, to obtain a compound of formula IIId, 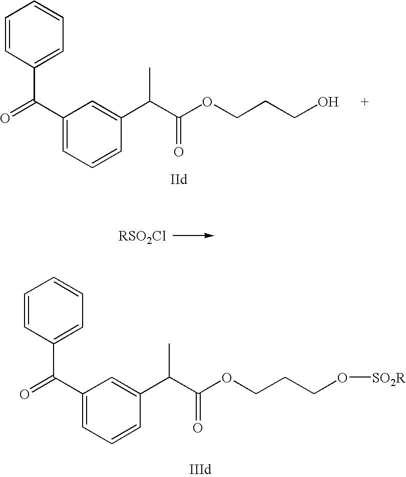
(3), reacting the compound of formula IIId with a nitrate source Y—NO₃, wherein Y is as defined in claim 1, to obtain a compound of formula IVd,

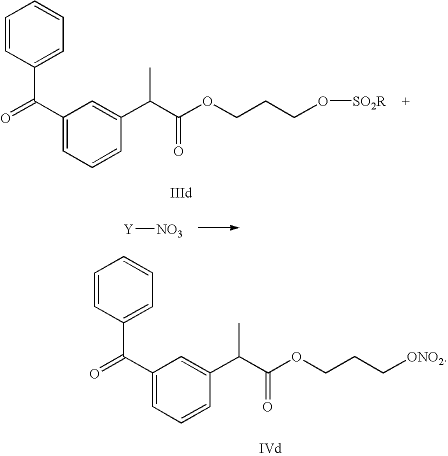

26. The process according to claim 25, wherein the compound of formula IVd is the S-enantiomer of NO donating ketoprofen.

27. 2-[2-(nitrooxy)ethoxy]-ethyl {2-[(2,6-dichlorophenyl)amino]phenyl}acetate (IVa) in a substantially crystalline form.

28. The compound according to claim 27 in anhydrous form.

29. The compound according to claim 27, characterized by the major peaks in the X-ray powder diffractogram shown below:

| D/Å | Relative |
|---|---|
| 12.7 | M |
| 8.7 | W |
| 8.1 | W |
| 6.3 | S |
| 5.94 | M |
| 5.91 | M |
| 5.58 | M |
| 5.34 | M |
| 5.05 | W |
| 4.50 | S |
| 4.48 | S |
| 4.38 | M |
| 4.35 | M |
| 4.28 | M |
| 4.23 | S |
| 4.08 | S |
| 4.06 | S |
| 3.96 | S |
| 3.78 | S |
| 3.76 | S |
| 3.55 | W |
| 3.52 | M |
| 3.49 | M |
| 3.44 | W |
| 3.41 | VS |
| 3.31 | W |
| 3.28 | M |
| 3.17 | S |
| 3.15 | S |
| 3.13 | W |
| 3.06 | M |
| 3.04 | W |
| 2.97 | M |
| 2.96 | M |
| 2.81 | W |
| 2.70 | M |
| 2.68 | M |
| 2.64 | M |
| 2.60 | W |
| 2.54 | W |
| 2.43 | W |

30. The compound according to claim 27, characterized by having a monoclinic unit cell with parameters a=13.79 Å, b=11.90 Å, c=13.01 Å, α=90°, β=94.0°, γ=90°.

31. A process for the production of Form A of compound IVa comprising crystallizing 2-[2-(nitrooxy)ethoxy]ethyl {2-[(2,6-dichlorophenyl)amino]phenyl}acetate.

32. Compounds of formula IIIa, IIIb, IIIc and IIId: 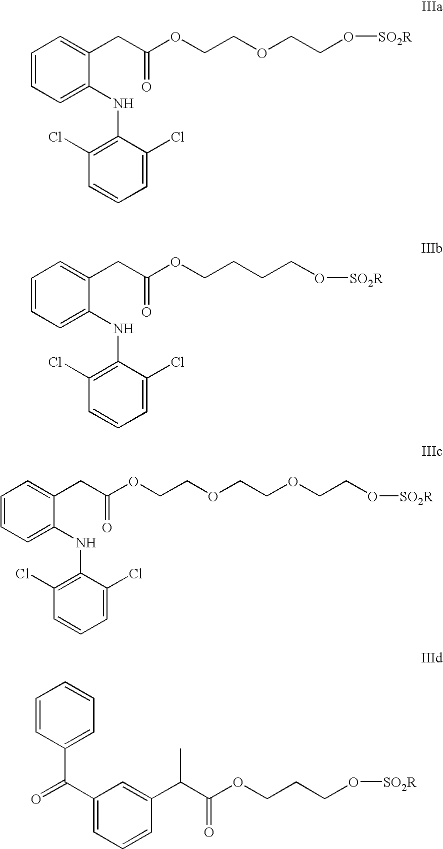
wherein R is selected from the group consisting of C₁-C₈ alkyl, phenyl, phenylmethyl, C₁-C₄ alkylphenyl, halophenyl, nitrophenyl, acetylaminophenyl, halogen, CF₃, and n-C₄F₉.

33. Use of the process according to claim 1 for the large scale manufacturing of the compounds of formula IVa, IVb, IVc and IVd.

34. Use of the compounds of formula III, ML_{T1}A_{T2}—X—O—SO₂R, wherein M, L, A, T1, T2, X and R are as defined in claim 1, as an intermediate for the manufacturing of a pharmaceutically active compound.

35. Use of intermediate compounds of formula IIIa, IIIb, IIIc and IIId as defined in claim 32, prepared according to the process described under step 1 and 2 of claim 1, for the manufacturing of a medicament for the treatment of pain and/or inflammation.

36. Use of Form A of compound IVa for the manufacturing of a medicament.

37. Use of Form A of compound IVa for the manufacturing of a medicament for the treatment of pain and/or inflammation.

38. A pharmaceutical formulation comprising a therapeutically effective amount of Form A of compound IVa, optionally in association with diluents, excipients or carriers.
